(19) United States
(12) Patent Application Publication
(10) Pub. No.: US 2012/0201750 A1
(43) Pub. Date: Aug. 9, 2012

(54) SERUM BIOMARKERS FOR MELANOMA METASTASIS

(76) Inventor: Bungwoo Ryu, Las Vegas, NV (US)

(21) Appl. No.: 13/237,377

(22) Filed: Sep. 20, 2011

Related U.S. Application Data
(60) Provisional application No. 61/384,619, filed on Sep. 20, 2010.

(51) Int. Cl. A61K 51/10; C40B 30/04; G01N 33/574; A61K 38/20; A61K 38/21; A61K 31/713; A61K 33/24; A61K 39/395; A61K 31/4375; A61P 35/00; A61K 31/7088; A61K 38/02; C12Q 1/68; A61K 39/00

(52) U.S. Cl. 424/1.49; 435/6.11; 506/9; 435/7.1; 424/85.2; 424/85.7; 424/85.4; 424/277.1; 424/649; 424/172.1; 424/133.1; 514/283; 514/44 R; 514/19.3; 514/44 A

(57) ABSTRACT

The present invention relates to methods for predicting and evaluating metastasis of solid cancers, such as melanoma, in a subject by measuring serum biomarkers associated with a metastatic phenotype. In particular, the present invention provides a serum gene expression signature that is different between highly aggressive and more metastatic versus less aggressive and less metastatic melanomas by quantitatively measuring the levels of, inter alia, lymphoid-specific helicase (HELLS) and condensing complex subunit 2 (NCAPH) transcripts in a subject.

### INCORPORATION BY REFERENCE

This application claims priority to U.S. Provisional Application Ser. No. 61/384,619, filed on Sep. 20, 2010.

Each of the applications and patents cited in this text, as well as each document or reference cited in each of the applications and patents (including during the prosecution of each issued patent; “application cited documents”), and each of the U.S. and foreign applications or patents corresponding to and/or claiming priority from any of these applications and patents, and each of the documents cited or referenced in each of the application cited documents, are hereby expressly incorporated herein by reference. More generally, documents or references are cited in this text, either in a Reference List before the claims, or in the text itself; and, each of these documents or references (“herein-cited references”), as well as each document or reference cited in each of the herein-cited references (including any manufacturer's specifications, instructions, etc.), is hereby expressly incorporated herein by reference. Documents incorporated by reference into this text may be employed in the practice of the invention.

### GOVERNMENT INTEREST STATEMENT

This invention was made with government support under grant number NIH K01 CA113779 awarded by the National Institutes of Health, Nevada Cancer Institute (NVCI) Startup Grant, and DOE DE-FG01-08ER64608 grant awarded by the Department of Energy. The government has certain rights in the invention.

### FIELD OF THE INVENTION

The present invention relates generally to methods for detecting and/or diagnosing solid cancers, such as melanoma in a subject, comprising the use of biomarkers. In particular, the present invention involves quantitative measurement of biomarkers such as, inter alia, lymphoid-specific helicase (HELLS) and condensin complex subunit 2 (NCAPH) gene transcripts in bodily fluids, which provide valuable diagnostic and prognostic information for predicting and evaluating the aggressiveness, including metastatic potential, of solid cancers in patients.

### BACKGROUND OF THE INVENTION

Metastasis of tumor from a primary site to distant organs poses a significant medical problem and is the leading cause of human cancer deaths [Mehlen, P., and Puisieux, A. (2006). Metastasis: a question of life or death. Nature Rev. 6, 449-58)]. Metastatic tumors are highly refractory to therapeutic treatments and typically result in poor clinical outcomes. For example, whereas early melanoma patients [American Joint Committee on Cancer (AJCC) Stage I and II] have a 10-year survival rate of approximately 90%, the 5-year survival rate for AJCC Stage IV patients is less than 5% [Balch, C. M., Gershenwald, J. E., Soong, S. J., Thompson, J. F., Atkins, M. B., Byrd, D. R., Buzaid, A. C., Cochran, A. J., Coit, D. G., Ding, S., et al. (2009). Final version of 2009 AJCC melanoma staging and classification. J. Clin. Oncol. 27, 6199-206).]. Histologic examination of early melanoma lesions is difficult and is not always predictive for melanoma aggressiveness [Cook, M. G., Spatz, A., Brocker, E. B., and Ruiter, D. J. (2002). Identification of histological features associated with metastatic potential in thin (<1.0 mm) cutaneous melanoma with metastases. A study on behalf of the EORTC Melanoma Group. J. Pathol. 197, 188-93)]. Breslow thickness and ulceration remain the most powerful prognostic factors for primary melanomas. However, these features are not completely accurate biological indicators for malignant potential as a subgroup of patients with a thin localized melanoma (<2.0 mm) eventually develop metastasis [Becker, D., Mihm, M. C., Hewitt, S. M., Sondak, V. K., Fountain, J. W., and Thurin, M. (2006). Markers and tissue resources for melanoma: meeting report. Cancer Res. 66, 10652-7; Slingluff, C. L., Jr., and Seigler, H. F. (1992). “Thin” malignant melanoma: risk factors and clinical management. Annals Plastic Surg. 28, 89-94)]. Discovery of reliable biomarkers that could identify melanoma patients with metastasis as early as possible for adjuvant treatment will contribute to increased survival.

The invention described herein is based on the identification of a population of circulating nucleic acids (CNAs), including in particular gene transcripts, whose detection in serum at elevated levels versus control subjects are likely to predict disease progression. Thus, patients with a history of melanoma who are at high risk for disease recurrence may be monitored for disease using a simple, readily-available blood test. Since the gene transcripts identified are specifically upregulated in aggressive melanomas, including metastasizing melanomas, which are melanomas that lead to the greatest mortality, it is expected that many of these “aggressive melanoma genes” will function as effective therapeutic targets for invasive melanomas. Accordingly, new tumor biomarkers and therapies can be developed based on their relevance to disease onset and progression.

Described herein are biomarkers for melanoma status, where the biomarkers can be correlated with a stage or a status of melanoma progression. The biomarkers of the present invention provide a way to correlate expression with disease state, thus providing diagnostic, prognostic and therapeutic potential that was heretofore unrecognized.

### SUMMARY OF THE INVENTION

The present invention describes a set of biomarkers that are differentially present as transcripts in a sample or samples of subjects with aggressive, metastatic melanoma versus the samples of control subjects or subjects with less aggressive, non-metastatic melanoma. Such biomarkers are found in samples from a subject, which include bodily fluids such as, but not limited to, serum, blood, blood plasma, urine, sputum, seminal fluid, cerebrospinal fluid and the like. The measurement of these biomarkers, alone or in combination, in subject samples provides information that one using relatively non-invasive methods can not only use to determine a probable diagnosis of melanoma or a negative diagnosis (e.g., normal or disease-free), but can also use to provide information about the stage of melanoma present. The present invention also provides methods and kits that are useful in diagnostic assays for determining disease status or stage in subjects diagnosed with, suspected of having, or at risk for developing melanoma, recurrence of melanoma, or metastasis of melanoma by measuring these biomarkers in samples of bodily fluids. In addition, these biomarkers can be of use in screening for disease onset/stage of disease/progression of disease in subjects with a known history of melanoma and those at risk for melanoma.

In a first aspect, the invention provides a serum biomarker for melanoma comprising HELLS and/or NCAPH RNA sequences, optionally in combination with TYMS and/or BIRC5 RNA sequences, wherein the biomarker is correlated with the presence of melanoma.

In a first embodiment, the melanoma is one or more of in situ, radial growth phase, vertical growth phase, and metastatic melanoma.

In another embodiment, the biomarker is further used to determine the metastatic potential or stage of a melanocyte tumor of undetermined malignant potential, or a tumor of undetermined classification.

In another related embodiment, the marker corresponds to a gene involved in a biological process selected from the group consisting of: DNA replication initiation, regulation of apoptosis or cell proliferation.

In certain embodiments, the melanoma is recurrent. In one embodiment, the biomarker is associated with aggressive melanoma.

In another aspect, the invention features a serum biomarker for melanoma status comprising HELLS and/or NCAPH RNA sequences, optionally in combination with TYMS and/or BIRC5 RNA sequences, wherein the biomarker is correlated with a stage of melanoma progression.

In another aspect, the invention features a biomarker for melanoma status comprising HELLS and/or NCAPH RNA sequences, optionally in combination with TYMS and/or BIRC5 RNA sequences, wherein the biomarker is correlated with invasive or metastatic melanoma.

In one particular embodiment, one or more of the biomarkers is overexpressed, and the overexpression is correlated with invasive or metastatic melanoma.

In one aspect, the invention features a method of detecting or diagnosing melanoma in a subject comprising (a) measuring at least one biomarker in a sample from the subject, wherein the biomarker is selected from the group consisting of HELLS, NCAPH, TYMS and BIRC5; and (b) correlating the measurement of the biomarker with melanoma status, thereby detecting or diagnosing melanoma in the subject.

The sample can be whole blood, serum, plasma, lymphatic fluid, interstitial fluid, cerebrospinal fluid (CSF), seminal fluid, saliva, mucous, sputum, sweat, or urine.

In one embodiment, measuring comprises detecting the presence or absence of the biomarker, quantifying the amount of the biomarker, and qualifying the type of the biomarker.

The melanoma can comprise in situ, radial growth phase, vertical growth phase, recurrent melanoma, or metastatic melanoma.

In one embodiment, the biomarker is HELLS. In another embodiment, the biomarker is NCAPH. The methods of the invention also concerns measurement of at least two biomarkers, at least three biomarkers, or all four biomarkers HELLS, NCAPH, TYMS, and BIRC5.

The biomarkers can be detected by any method known to those skilled in the art, such as, e.g., polymerase chain reaction (PCR), microarray analysis, or immunoassay. Preferably, the biomarkers are detected by quantitative real-time RT-PCR.

In another embodiment, the methods of the invention further comprise managing subject treatment based on melanoma status in the subject. Managing subject treatment can be selected from ordering further diagnostic tests, administering at least one therapeutic agent, surgery, surgery followed or preceded by administering at least one therapeutic agent, biotherapy, and taking no further action. The therapeutic agent can be selected from one or more of fotemustine, dacarbazine, interferon, cisplatin, tamoxifen, interleukin-2, interferon alpha, vinblastin, carmubris, avastin, BRAF-kinase inhibitor, CTLA-4 antibody, angiogenesis inhibitors, targeted immunotherapy, or vaccines.

In another embodiment, the methods of the invention further comprise measuring the biomarker after managing subject treatment.

Melanoma status according to the methods described herein can be selected from one or more of the presence, absence, or amount of the biomarker. Melanoma status may be assessed by one or more of visual examination, tissue sample examination, subject's symptoms, or blood evaluation.

The subject may be one who has been previously diagnosed with melanoma. Alternatively, the subject may be one who has not been previously diagnosed with melanoma. The subject may also be one who has previously been treated for melanoma.

In another embodiment, the measurement is used to predict the recurrence of melanoma, or to classify the subject as a low or high risk for melanoma recurrence.

In other embodiments, the methods of the invention involve correlation that may be performed by a software classification algorithm. The methods of the invention may further comprise correlating the measurement of the biomarker with a melanoma stage. The melanoma stage may be Stage IV melanoma.

In another aspect, the invention features a method of determining prognosis of a subject suffering from melanoma comprising: (a) measuring at least one biomarker in a sample from the subject, wherein the biomarker is selected from the group consisting of HELLS, NCAPH, TYMS and BIRC5; and (b) correlating the measurement with prognosis of melanoma, thereby determining the prognosis of the subject suffering from melanoma.

In one embodiment, the prognosis determines course of treatment.

In another aspect, the invention features a method of treating melanoma comprising administering to a subject suffering from or at risk of developing melanoma a therapeutically effective amount of a compound capable of modulating the expression or activity of at least one biomarker selected from the group consisting of: HELLS, NCAPH, TYMS and BIRC5 in the subject.

In yet another aspect, the invention features a method of treating a condition in a subject comprising administering to a subject a therapeutically effective amount of a compound which modulates the expression of one or more of the genes HELLS, NCAPH, TYMS and/or BIRC5.

In one embodiment, the compound directly modulates the expression of one or more of the HELLS, NCAPH, TYMS and/or BIRC5 genes.

In one embodiment, the compound is selected from the group consisting of enzyme inhibitors, cytotoxic drugs, cytokines, chemokines, antibodies, a DNA molecule, an RNA molecule, a small molecule, a peptide, and a peptidomimetic. In another embodiment, the compound is an antibody. The antibody can be selected from the group consisting of: monoclonal, polyclonal, humanized, and chimeric antibodies. In a particular embodiment, the antibody is radiolabeled.

In other embodiments, the compound is an inhibitory RNA molecule, which can be one or more siRNAs. The siRNA may be of any length, but is preferably about 18-21 nucleotides in length.

In another aspect, the invention features a method of determining melanoma status of a subject, comprising: (a) obtaining a biomarker profile from a sample taken from the subject; and (b) comparing the subject's biomarker profile to a reference biomarker profile obtained from samples of a reference population, wherein the comparison is capable of classifying the subject as belonging to or not belonging to the reference population; wherein the subject's biomarker profile and the reference biomarker profile comprise at least one biomarker selected from the group consisting of: HELLS, NCAPH, TYMS and BIRC5.

In one embodiment, the method further comprises repeating the method at least once, wherein the subject's biomarker profile is obtained from a separate sample taken each time the method is repeated. In a related embodiment, the samples from the subject are taken about 24 hours apart. In another embodiment, the comparison of the biomarker profiles can determine melanoma status in the subject with an accuracy of at least about 60% to about 99%. In another embodiment, the reference biomarker profile is obtained from a population comprising a single subject, at least two subjects, and at least 20 subjects.

In another related embodiment, the invention features a purified biomolecule selected from the HELLS and/or the NCAPH RNA sequences, optionally in combination with the TYMS RNA sequences and/or the BIRC5 RNA sequences.

In another aspect, the invention features a method of qualifying cancer status in a subject comprising (a) measuring at least one biomarker selected from the group consisting of: HELLS, NCAPH, TYMS and BIRC5; and (b) correlating the measurement with cancer status, thereby qualifying cancer status in the subject.

In another embodiment, the biomarker is one or both of HELLS transcript or NAPCH transcript. In a particular embodiment, the cancer is a solid tumor. In a particular embodiment, the cancer is a hematological malignancy.

In another aspect, the invention features a kit for detecting or diagnosing melanoma, comprising one or more reagents for detecting at least one biomarker selected from the group consisting of: HELLS, NCAPH, TYMS and BIRC5, and written instructions for use of the kit for detection or diagnosis of melanoma. In another embodiment, the instructions provide for contacting a sample from a subject with the reagents and detecting the biomarkers. The reagents may comprise an adsorbent that retains the biomarkers, which may be a single or double stranded oligonucleotide. The biomarker may be detected by any means known to the skilled artisan, such as, e.g., nucleic acid sequencing, PCR, or immunoassay, preferably quantitative real-time RT-PCR.

In another aspect, the invention features a biomarker for cancer status comprising the biomarkers comprising HELLS and/or NCAPH RNA sequences, optionally in combination with TYMS and/or BIRC5 RNA sequences, wherein the biomarker is correlated with cancer status.

In a related embodiment, the biomarker is HELLS transcript. In a related embodiment, the biomarker is NCAPH transcript.

In another embodiment, the cancer is a solid tumor. In a further embodiment, the cancer is a hematological malignancy.

In another aspect, the invention features a method of qualifying cancer status in a subject comprising (a) measuring the biomarker HELLS and/or NCAPH RNA sequences, optionally in combination with TYMS and/or BIRC5 RNA sequences; and (b) correlating the measurement with cancer status, thereby qualifying cancer status in a subject.

In another embodiment, the biomarker is one or both of HELLS transcript or NAPCH transcript. In a particular embodiment, the cancer is a solid tumor. In a particular embodiment, the cancer is a hematological malignancy.

Unless otherwise defined, all technical and scientific terms used herein have the same meaning as commonly understood by one of ordinary skill in the art to which this invention pertains. Although methods and materials similar or equivalent to those described herein can be used in the practice of the present invention, suitable methods and materials are described below. All publications, patent applications, patents, and other references mentioned herein are expressly incorporated by reference in their entirety. In cases of conflict, the present specification, including definitions, will control. In addition, the materials, methods, and examples described herein are illustrative only and are not intended to be limiting.

Other features and advantages of the invention will be apparent from the following detailed description and claims.

### BRIEF DESCRIPTION OF THE DRAWINGS

The following Detailed Description, given by way of example, but not intended to limit the invention to specific embodiments described, may be understood in conjunction with the accompanying Figures, incorporated herein by reference, in which:

FIG. 1 is a Table that shows a comparison of melanoma patient characteristics between AJCC stage 0/I and stage IV groups used in the supervised analysis serum samples.

FIG. 2 shows distribution patterns of circulating gene transcripts in patient sera and area under the receiver operating characteristic (AUC-ROC) curves. (A) Comparison of quantitative mRNA copy number for the genes HELLS, NCAPH, TYMS, BIRC5, and ACTB, in patient sera among the sample groups (N: normal control, P: patients with Stage 0/1 primary melanomas, M: patients with Stage IV metastatic melanomas). ACTB serves as a negative control. (B) Comparison of serum LDH concentrations in the patients with primary and metastatic melanomas. (C) AUC-ROC curves for each individual analyte are shown.

FIG. 3 is a Table that shows ANOVA results comparing individual gene transcript between the local and metastatic groups.

FIG. 4 is a Table summarizing a logistic regression analysis predicting the probability of each gene transcript with respect to the presence of metastatic melanomas.

### DETAILED DESCRIPTION OF THE INVENTION

The present invention is based in part on a study that showed that circulating transcripts could be useful in evaluating metastatic potential of melanomas. The findings indicate that one or more of a group of circulating metastasis-associated gene transcripts (HELLS, NCAPH, TYMS, and BIRC5), particularly HELLS gene transcript, can be useful for determining or diagnosing melanoma, including the risk of metastasis in a subject.

Using a global gene expression profiling technique, gene signatures associated with metastatic phenotype was discovered in a panel of melanoma cell lines derived from histopathologically well-characterized lesions of melanomas [Ryu, B., Kim, D. S., Deluca, A. M., and Alani, R. M. (2007). Comprehensive expression profiling of tumor cell lines identifies molecular signatures of melanoma progression. PloS One 2, e594; Alani, R. M., Ryu, B., and Keefe, M. J. (2008) Biomarkers for Melanoma., International Application Publication No. WO/2008/141275. Winnepenninckx et al. also reported a 254-gene signature associated with high-risk of recurrent metastasis and poor clinical outcomes in a study using human tissue samples [Winnepenninckx, V., Lazar, V., Michiels, S., Dessen, P., Stas, M., Alonso, S. R., Avril, M. F., Ortiz Romero, P. L., Robert, T., Balacescu, O., et al. (2006). Gene expression profiling of primary cutaneous melanoma and clinical outcome. J. Natl. Cancer Inst. 98, 472-82]. Significant overlap was found in these gene signatures [Van Den Oord, J. J., Sarasin, A., Winnepenninckx, V., and Spatz, A. (2007). Expression profiling of melanoma cell lines: in search of a progression-related molecular signature. Future Oncol. 3, 609-11]. Common molecular pathways mediated by these signature genes in metastatic melanomas are activation of DNA replication origins, DNA repair and mitogenic proliferation [Kauffmann, A., Rosselli, F., Lazar, V., Winnepenninckx, V., Mansuet-Lupo, A., Dessen, P., Van Den Oord, J. J., Spatz, A., and Sarasin, A. (2008). High expression of DNA repair pathways is associated with metastasis in melanoma patients. Oncogene 27, 565-73]. These findings indicate that one of the distinctive molecular characteristics of metastatic melanoma is the overexpression of genes associated with these cellular pathways.

One limitation to the use of biomarkers to predict tumor aggressiveness and metastatic potential is the requirement for clinical sampling. A non-invasive approach to evaluate tumor status is via analysis of bodily fluid samples from a subject, including, but not limited to, blood and serum. There are at least two ways in which genetic signatures can be obtained from blood of cancer patients. One is by obtaining and analyzing rare, circulating cancer cells [e.g., Peck, K., Sher, Y.-P., Shih, J.-Y., Roffler, S. R., Wu, C.-W., and Yang, P.-C. (1998). Detection and Quantitation of Circulating Cancer Cells in the Peripheral Blood of Lung Cancer Patients. Cancer Res. 58, 2761-5]. Alternatively, the difficult process of isolating the small number of circulating tumor cells from the larger number of resident blood cells can be eliminated by direct measurement of cell-free, circulating nucleic acid (CNA), including, but not limited to, gene transcripts. It has been known for more than 30 years that the plasma of cancer patients contains both RNA and DNA and evidence accumulated over the intervening years supports the view that CNAs were indicative of tumor-related alterations [reviewed by Bremnes, R. M., Sirera, R., and Camps, C. (2005). Circulating tumour-derived DNA and RNA markers: a tool for early detection, diagnostics and follow-up? Lung Cancer 49, 1-12]. CNAs are useful to predict not only the presence or absence of cancer in a patient, but also provide guidance regarding some of the characteristics of the cancer. For example, the presence of certain gene transcripts in a patient's blood was found to correlate with the disease stage and/or degree of aggressiveness. As a further non-limiting example, a subpopulation of CNAs that are associated with metastatic cancer and quantitation of such CNAs were found to be useful as biomarkers for occurrence of metastases.

CNA consists of extracellular genetic material. Tumor-derived CNAs have great potential as a noninvasive source of candidate biomarkers because they can be obtained for monitoring the status of remote tumors by decoding the contained genetic and epigenetic information without the need for tissue biopsies [Swarup, V., and Rajeswari, M. R. (2007). Circulating (cell-free) nucleic acids—a promising, non-invasive tool for early detection of several human diseases. FEBS Lett. 581, 795-9; Taback, B., and Hoon, D. S. (2004). Circulating nucleic acids in plasma and serum: past, present and future. Curr. Opin. Mol. Therapeutics. 6, 273-8]. One of the most important factors determining the potential value of CNA as a predictive biomarker is the molecular information intrinsically encoded in the target CNAs. For example, genetic/epigenetic changes in genes specific for the clinicopathologic characteristics of tumors as well as differential levels of circulating mRNAs characteristic of genes that have functional roles in the malignant progression were found to be good CNA candidates as a source of predictive biomarkers.

The present invention provides biomarkers for melanoma status, wherein the biomarkers are correlated with the progression or stage of melanoma. In particular, the invention provides biomarkers that may be used to detect, diagnose, or monitor disease onset or progression in patients with known previous diagnosis of melanoma and in patients at high risk for the development of melanoma. These progression-associated genes are useful as diagnostic/prognostic biomarkers of melanoma, as well as novel therapeutic targets. The invention provides that these biomarkers, used individually, or in combination with other biomarkers from this group or with other diagnostic tests, provide a novel method of determining, detecting, or diagnosing melanoma status or prognosis in a subject, and correlating the status of melanoma to the progression or stage of disease.

The biomarkers of the invention are differentially present in samples of melanoma subjects and control subjects (i.e., subjects in whom melanoma is undetectable), and are differentially present at different stages of melanoma progression. Some of these biomarkers are found at an elevated level and/or more frequently in samples from human melanoma subjects compared to a control (e.g., subjects with diseases other than melanoma). Accordingly, the amount of one or more biomarkers found in a test sample compared to a control, or the mere detection of one or more biomarkers in the test sample provides useful information regarding probability of whether a subject being tested has melanoma or not, and/or whether a subject being tested has a particular melanoma subtype or not. Thus, the present invention relates to methods and kits for determining or diagnosing melanoma status and prognosis. In particular, the detection of these biomarkers is particularly useful for detecting recurrent melanoma in a patient that has a high risk for recurrent disease.

The identified melanoma biomarkers of the invention are useful to predict disease progression. In one embodiment, subjects having a history of melanoma who are at high risk for disease recurrence may be monitored for disease using the methods and kits described herein. Current disease monitoring is achieved through the use of frequent physical examinations in conjunction with various imaging modalities including CT-scanning, MRI scanning, and PET scanning. Such subject monitoring techniques often detect only grossly-detectable disease which is often difficult to treat. The claimed methods and kits fulfill a long-felt need in the art for earlier detection of disease recurrence/progression and therefore earlier treatment of subjects with recurrent/progressive disease.

“Accuracy” refers to the degree of conformity of a measured or calculated quantity (a test reported value) to its actual (or true) value. Clinical accuracy relates to the proportion of true outcomes (true positives (TP) or true negatives (TN) versus misclassified outcomes (false positives (FP) or false negatives (FN)), and may be stated as a sensitivity, specificity, positive predictive values (PPV) or negative predictive values (NPV), or as a likelihood, odds ratio, among other measures. Preferably, the accuracy of determining melanoma status in a subject is at least about 60%, at least about 70%, at least about 80%, at least about 90%, at least about 95%, at least about 99% accurate and any increment thereof approaching 100%.

A “biomarker” in the context of the present invention is a molecular indicator of a specific biological property; a biochemical feature or facet that can be used to measure the progress of disease or the effects of treatment. “Biomarker” encompasses, without limitation, nucleic acids, such as, e.g., circulating nucleic acids (CNA), including their polymorphisms, mutations, variants, modifications, subunits, fragments complexes, and degradation products. Biomarkers can also include mutated proteins or mutated nucleic acids. A combination of biomarkers, or “profile” can comprise a validated selection of optimal biomarkers. Selection of an effective set of optimal biomarkers involves differentiating which genes are particularly indicative of melanoma and/or melanoma metastasis.

“Detect” or “detection” refers to identifying the presence, absence or amount of the object to be detected.

The term “differentially present” refers to differences in the quantity and/or the frequency of a biomarker present in a sample taken from subjects having melanoma as compared to a control subject or a reference subject or sample. For example, some of the biomarkers described herein are present at an elevated level in samples of subjects compared to samples from control subjects. Furthermore, a biomarker as used herein can be a nucleic acid which is detected at a higher frequency in samples of human melanoma subjects compared to samples of control subjects.

For example, a nucleic acid is differentially present between two samples if the amount of the nucleic acid in one sample is statistically significantly different from the amount of the nucleic acid in the other sample. For example, a nucleic acid is differentially present between the two samples if it is present at least about 50%, at least about 75%, at least about 100%, 120%, at least about 130%, at least about 150%, at least about 180%, at least about 200%, at least about 300%, at least about 500%, at least about 700%, at least about 900%, or at least about 1000% greater than it is present in the other sample, or if it is detectable in one sample and not detectable in the other.

Alternatively or additionally, a nucleic acid is differentially present between two sets of samples if the frequency of detecting the nucleic acid in the melanoma subjects' samples is statistically significantly higher than in the control samples. For example, a polypeptide or nucleic acid is differentially present between the two sets of samples if it is detected at least about 50%, at least about 75%, at least about 100%, at least about 120%, at least about 130%, at least about 150%, at least about 180%, at least about 200%, at least about 300%, at least about 500%, at least about 700%, at least about 900%, or at least about 1000% more frequently or less frequently observed in one set of samples than the other set of samples.

A “formula,” “algorithm,” or “model” is any mathematical equation, algorithmic, analytical or programmed process, or statistical technique that takes one or more continuous or categorical inputs (herein called “parameters”) and calculates an output value, sometimes referred to as an “index” or “index value.” Non-limiting examples of “algorithms” include sums, ratios, and regression operators, such as coefficients or exponents, biomarker value transformations and normalizations (including, without limitation, those normalization schemes based on clinical parameters, such as gender, age, smoking status, or ethnicity), rules and guidelines, statistical classification models, and neural networks trained on historical populations. Of particular use in combining the biomarkers of the present invention are linear and non-linear equations and statistical classification analyses to determine the relationship between levels of biomarkers detected in a subject sample and the subject's risk of cancer, particularly solid tumors.

For complex statistical data analysis derived from the disclosed composition and methods, Principal Component Analysis (PCA) can be generally applied, however any algorithm or computed index can be used, such as but not limited to, cross-correlation, factor rotation, Logistic Regression (LogReg), Linear Discriminant Analysis (LDA), Eigengene Linear Discriminant Analysis (ELDA), Support Vector Machines (SVM), Random Forest (RF), Recursive Partitioning Tree (RPART), as well as other related decision tree classification techniques, Shrunken Centroids (SC), StepAIC, Kth-Nearest Neighbor, Boosting, Decision Trees, Neural Networks, Bayesian Networks, Support Vector Machines, Leave-One-Out (LOO), 10-Fold cross-validation (10-Fold CV), and Hidden Markov Models, among others.

“Managing subject treatment” refers to the behavior of the subject, clinician or physician subsequent to the determination of melanoma status or prognosis. For example, if the result of the methods of the present invention is inconclusive or there is reason that confirmation of status is necessary, the physician may order more diagnostic tests. Alternatively, if the status indicates that treatment is appropriate, the physician may schedule the subject for treatment, e.g., surgery, administer one or more therapeutic agents, biological therapies, or radiation. Surgery may follow or precede administration of at least one therapeutic agent. Likewise, if the status is negative, e.g., late stage melanoma or if the status is acute, no further action may be warranted. Furthermore, if the results show that treatment has been successful, a maintenance therapy or no further management may be necessary. The invention also encompasses carrying out any of the methods described herein (i.e., measurement of one or more biomarkers) after managing subject treatment.

“Measuring” or “measurement” means assessing the presence, absence, quantity or amount (which can be an effective amount) of either a given substance within a clinical or subject-derived sample, including the derivation of qualitative or quantitative concentration levels of such substances, or otherwise evaluating the values or categorization of a subject's clinical parameters. Measurement or measuring may also involve qualifying the type or identifying the biomarker(s). Measurement of the biomarkers of the invention may be used to diagnose, detect, or identify melanoma (or metastasis of melanoma) in a subject, to monitor the progression or prognosis of melanoma (or metastasis of melanoma) in a subject, to predict the recurrence of melanoma (or metastasis of melanoma) in a subject, or to classify a subject as having a low risk or a high risk of developing melanoma (or metastasis of melanoma) or a recurrence of melanoma.

“Melanoma stage” is determined or diagnosed in the art using the “tumor-node-metastasis” system. Stage 0 melanoma involves the melanoma that is present in the epidermis but has not reached the underlying dermis. This stage is also called “melanoma in situ” (TisN0M0). Stage I melanoma is characterized by tumor thickness, presence and number of mitoses, and ulceration status, without regional lymph node or distant metastasis. There are two subclasses of Stage I melanoma: Stage IA, characterized by “T1aN0M0” (tumor less than or equal to 1 mm, no ulceration, and no mitoses) and Stage IB, which is characterized by “T1bN0M0” or “T2aN0M0” (tumor less than or equal to 1 mm, with ulceration or mitoses). Stage II melanoma is also characterized by tumor thickness and ulceration status with no evidence of regional lymph node or distant metastasis. The three subclasses of Stage II melanoma include: Stage IIA (T2bN0M0 or T3aN0M0), Stage IIB (T3bN0M0 or T4aN0M0) and Stage IIC (T4bN0M0). Stage III melanoma is characterized by the level of lymph node metastasis with no evidence of distant metastasis. The three subclasses of Stage III melanoma include: Stage IIIA (T1-T4a N1aM0 or T1-T4aN2aM0), Stage IIIB (T1-T4bN1aM0, T1-T4bN2aM0, T1-T4aN1bM0, T1-T4aN2bM0, or T1-T4a/bN2cM0), and Stage IIIC (T1-4-bN1bN0, T1-4-bN2bM0, or T1-4a/bN3M0). Stage IV melanoma is characterized by the location of distant metastases and the level of serum lactate dehydrogenase (LDH). Stage IV melanomas include any T or N classification.

The term “melanoma status” refers to the status of the disease in the subject. Examples of types of melanoma statuses include, but are not limited to, the subject's risk of melanoma, the presence or absence of disease, the stage of disease in a subject (e.g., stages 0-IV and recurrent melanoma), and the effectiveness of treatment of disease. Melanoma status may refer to in situ disease or invasive disease. Other statuses and degrees of each status are known in the art.

“Risk” in the context of the present invention relates to the probability that an event will occur over a specific time period, as in the development or growth or metastasis of melanoma, and can mean a subject's “absolute” risk or “relative” risk. Absolute risk can be measured with reference to either actual observation post-measurement for the relevant time cohort, or with reference to index values developed from statistically valid historical cohorts that have been followed for the relevant time period. Relative risk refers to the ratio of absolute risks of a subject compared either to the absolute risks of low risk cohorts or an average population risk, which can vary by how clinical risk factors are assessed. Odds ratios, the proportion of positive events to negative events for a given test result, are also commonly used (odds are according to the formula p/(1−p) where p is the probability of event and (1−p) is the probability of no event) to no-conversion. Alternative continuous measures which may be assessed in the context of the present invention include time to development of melanoma, or progression to a different stage of melanoma, including progression or development of metastatic melanoma and therapeutic cancer conversion risk reduction ratios.

“Risk evaluation,” or “evaluation of risk” in the context of the present invention encompasses making a prediction of the probability, odds, or likelihood that an event or disease state may occur, the rate of occurrence of the event or conversion from one disease state to another, i.e., from a “normal” condition to an at-risk condition for developing melanoma, or from an at-risk condition to melanoma, or development of recurrent melanoma. Risk evaluation can also comprise prediction of other indices of melanoma, either in absolute or relative terms in reference to a previously measured population. The methods of the present invention may be used to make continuous or categorical measurements of the risk of conversion to melanoma, thus diagnosing and defining the risk spectrum of a category of subjects defined as at risk for developing melanoma. In the categorical scenario, the invention can be used to discriminate between normal and at-risk subject cohorts. In other embodiments, the present invention may be used so as to discriminate at-risk conditions from cancerous conditions, or cancerous conditions from normal. Such differing use may require different biomarker combinations in individual panel or profile, mathematical algorithm, and/or cut-off points, but be subject to the same aforementioned measurements of accuracy for the intended use.

A “sample” in the context of the present invention is a biological sample isolated from a subject and can include, by way of example and not limitation, whole blood, serum, plasma, lymphatic fluid, interstitial fluid (also known as “extracellular fluid” and encompasses the fluid found in spaces between cells, including, inter alia, gingival crevicular fluid), seminal fluid, cerebrospinal fluid (CSF), saliva, mucous, sputum, sweat, urine, or any other secretion, excretion, or other bodily fluids.

“Solid support” refers to a solid material which can be derivatized with, or otherwise attached to, a capture reagent. Exemplary solid supports include probes, microtiter plates, beads, and chromatographic resins. A similar term in the context of the present invention is “adsorbent surface”, which refers to a surface to which is bound an adsorbent (also called a “capture reagent” or an “affinity reagent”). An “adsorbent” is any material capable of binding an analyte (e.g., a target polypeptide or nucleic acid). “Chromatographic adsorbent” refers to a material typically used in chromatography. Chromatographic adsorbents include, for example, ion exchange materials, metal chelators (e.g., nitriloacetic acid or iminodiacetic acid), immobilized metal chelates, hydrophobic interaction adsorbents, hydrophilic interaction adsorbents, dyes, simple biomolecules (e.g., nucleotides, amino acids, simple sugars and fatty acids) and mixed mode adsorbents (e.g., hydrophobic attraction/electrostatic repulsion adsorbents). “Biospecific adsorbent” refers an adsorbent comprising a biomolecule, e.g., a nucleic acid molecule (e.g., an aptamer), a polypeptide, a polysaccharide, a lipid, a steroid or a conjugate of these (e.g., a glycoprotein, a lipoprotein, a glycolipid, a nucleic acid (e.g., DNA)-protein conjugate). In certain instances the biospecific adsorbent can be a macromolecular structure such as a multiprotein complex, a biological membrane or a virus. Examples of biospecific adsorbents are antibodies, receptor proteins and nucleic acids. Biospecific adsorbents typically have higher specificity for a target analyte than chromatographic adsorbents. “Adsorption” refers to detectable non-covalent binding of an analyte to an adsorbent or capture reagent.

By “statistically significant”, it is meant that the alteration is greater than what might be expected to happen by chance alone (which could be a “false positive”). Statistical significance can be determined by any method known in the art. Commonly used measures of significance include the p-value, which presents the probability of obtaining a result at least as extreme as a given data point, assuming the data point was the result of chance alone. A result is often considered highly significant at a p-value of 0.05 or less.

A “subject” in the context of the present invention is preferably a mammal. The mammal can be a human, non-human primate, mouse, rat, dog, cat, horse, or cow, but are not limited to these examples. Mammals other than humans can be advantageously used as subjects that represent animal models of cancer, such as nude mice. A subject can be male or female. A subject can be one who has been previously diagnosed or identified as having melanoma, and optionally has already undergone, or is undergoing, a therapeutic intervention or treatment for the melanoma. Alternatively, a subject can also be one who has not been previously diagnosed as having melanoma. For example, a subject can be one who exhibits one or more risk factors for melanoma, or a subject who does not exhibit risk factors for melanoma, or a subject who is asymptomatic for melanoma. A subject can also be one who is suffering from or at risk of developing melanoma, or who is suffering from or at risk of developing a recurrence of melanoma. A subject can also be one who is suffering from or at risk of developing metastatic melanoma. A subject can also be one who has been previously treated for melanoma, whether by administration of therapeutic agents, radiation therapy, surgery, or any combination of the foregoing.

Samples are collected from subjects to establish melanoma status. The subjects may be subjects who have been determined to have a high risk of melanoma based on their family history, a previous treatment, subjects with physical symptoms known to be associated with melanoma, subjects identified through screening assays (e.g., routine melanoma screening) or other techniques. Other subjects include subjects who have melanoma and the test is being used to determine the effectiveness of therapy or treatment they are receiving. Also, subjects may include healthy people who are having a test as part of a routine examination, or to establish baseline levels of the biomarkers. Samples may be collected from subjects who had been diagnosed with melanoma and received treatment to eliminate the melanoma, or perhaps are in remission.

The risk of melanoma can be detected by measuring an “effective amount” of the biomarkers of the present invention in a sample (e.g., a subject derived sample), and comparing the effective amounts to reference values, often utilizing mathematical algorithms or formulae in order to combine information from results of multiple individual biomarkers into a single measurement. Subjects identified as having an increased risk of melanoma can optionally be selected to receive treatment regimens or therapeutic interventions, such as administration of compounds such as “therapeutic agents” as defined herein, or implementation of surgical interventions (which may follow or precede administration of therapeutic agents or other therapies), biological therapies (“biotherapies”), or radiological therapies to prevent or delay the onset or recurrence of melanoma or metastasis of melanoma.

The amount of the biomarker can be measured in a test sample and compared to a “reference biomarker profile”, utilizing techniques such as reference limits, discrimination limits, or risk defining thresholds to define cutoff points and abnormal values for melanoma. The reference biomarker profile means the level of one or more biomarkers or combined biomarker indices typically found in a subject or reference population (which can include a single subject, at least two subjects, or any number of subjects including 20 subjects or more) not suffering from melanoma. Such reference biomarker profiles and cutoff points may vary based on whether a biomarker is used alone or in a formula combining with other biomarkers into a single value. Alternatively, the reference biomarker profile can be a database of biomarker patterns from previously tested subjects who did not convert to melanoma over a clinically relevant time horizon.

The present invention may be used to make continuous or categorical measurements of the risk of conversion to melanoma, thus diagnosing and defining the risk spectrum of a category of subjects defined as being at-risk for developing melanoma. In the categorical scenario, the methods of the present invention can be used to discriminate between normal and at-risk subject cohorts. In other embodiments, the present invention may be used so as to discriminate at-risk from cancerous, or cancerous from normal. Such differing use may require different biomarker combinations in individual panel or profile, mathematical algorithm, and/or cut-off points, but be subject to the same aforementioned measurements of accuracy for the intended use.

Identifying the at-risk subject enables the selection and initiation of various therapeutic interventions or treatment regimens in order to delay, reduce, or prevent that subject's conversion to melanoma. Levels of an effective amount of biomarker also allows for the course of treatment of melanoma to be monitored. In this method, a biological sample can be provided from a subject undergoing treatment regimens, e.g., therapeutic treatments, for melanoma. Such treatment regimens can include, but are not limited to, surgical intervention, radiological therapies, and treatment with therapeutic agents used in subjects diagnosed or identified with melanoma. If desired, biological samples are obtained from the subject at various time points before, during, or after treatment. For example, determining the melanoma status by comparison of a subject's biomarker profile to a reference biomarker profile can be repeated more than once, wherein the subject's biomarker profile can be obtained from a separate sample taken each time the method is repeated. Samples may be taken from the subject at defined time intervals, such as, e.g., 24 hours, 48 hours, 72 hours, or any suitable time interval as would be performed by those skilled in the art.

Levels of an effective amount of biomarker can then be determined and compared to a reference value, e.g. a control subject or population whose melanoma status is known, or an index value or baseline value. The reference sample or index value or baseline value may be taken or derived from one or more subjects who have been exposed to the treatment, or may be taken or derived from one or more subjects who are at low risk of developing melanoma, or may be taken or derived from subjects who have shown improvements in cancer risk factors as a result of exposure to treatment. Alternatively, the reference sample or index value or baseline value may be taken or derived from one or more subjects who have not been exposed to the treatment. For example, samples may be collected from subjects who have received initial treatment for melanoma and subsequent treatment for melanoma to monitor the progress of the treatment. A reference value can also comprise a value derived from risk prediction algorithms or computed indices from population studies such as those disclosed herein.

The biomarkers of the present invention can thus be used to generate a reference biomarker profile of those subjects who do not have melanoma, and would not be expected to develop melanoma. The biomarkers disclosed herein can also be used to generate a “subject biomarker profile” taken from subjects who have melanoma. The subject biomarker profiles can be compared to a reference biomarker profile to diagnose or identify subjects at risk for developing melanoma, to monitor the progression of disease, as well as the rate of progression of disease, and to monitor the effectiveness of melanoma treatment modalities or subject management. The reference and subject biomarker profiles of the present invention can be contained in a machine-readable medium, such as but not limited to, analog or digital tapes like those readable by a VCR, CD-ROM, DVD-ROM, USB flash media, among others. Such machine-readable media can also contain additional test results, such as, without limitation, measurements of clinical parameters and traditional laboratory risk factors. Alternatively or additionally, the machine-readable media can also comprise subject information such as medical history and any relevant family history. The machine-readable media can also contain information relating to other cancer risk algorithms and computed indices such as those described herein.

The biomarkers and method of the present invention can be used in the diagnosis, prognosis, monitoring, or treatment of melanoma, which can comprise in situ melanoma, radial growth phase melanoma, vertical growth phase melanoma, recurrent melanoma, or metastatic melanoma, but can also be used to detect, diagnose, monitor, or treat a wide variety of cancers, including but not limited to solid tumors (e.g., tumors of the head and neck, lung, breast, colon, colorectal, prostate, bladder, rectum, brain, gastric tissue, bone, ovary, thyroid, or endometrium), hematological malignancies (e.g., leukemias, lymphomas, myelomas), carcinomas (e.g. bladder carcinoma, renal carcinoma, breast carcinoma, colorectal carcinoma), or neuroblastoma. Non-limiting examples of these cancers include diffuse large B-cell lymphoma (DLBCL), T-cell lymphomas or leukemias, e.g., cutaneous T-cell lymphoma (CTCL), noncutaneous peripheral T-cell lymphoma, lymphoma associated with human T-cell lymphotrophic virus (HTLV), adult T-cell leukemia/lymphoma (ATLL), as well as acute lymphocytic leukemia, acute nonlymphocytic leukemia, acute myeloid leukemia, chronic lymphocytic leukemia, chronic myelogenous leukemia, Hodgkin's disease, non-Hodgkin's lymphoma, myeloma, multiple myeloma, mesothelioma, childhood solid tumors, neuroblastoma, retinoblastoma, glioma, Wilms' tumor, bone cancer and soft-tissue sarcomas, common solid tumors of adults such as head and neck cancers (e.g., oral, laryngeal and esophageal), genitourinary cancers (e.g., prostate, bladder, renal, uterine, ovarian, testicular, rectal and colon), lung cancer (e.g., small cell carcinoma and non-small cell lung carcinoma, including squamous cell carcinoma and adenocarcinoma), breast cancer, pancreatic cancer, skin cancers, stomach cancer, brain cancer, liver cancer, adrenal cancer, kidney cancer, thyroid cancer, basal cell carcinoma, squamous cell carcinoma of both ulcerating and papillary type, metastatic skin carcinoma, medullary carcinoma, osteo sarcoma, Ewing's sarcoma, veticulum cell sarcoma, and Kaposi's sarcoma. Also included are pediatric forms of any of the cancers described herein.

Differences in the genetic makeup of subjects can result in differences in their relative abilities to metabolize various drugs, which may modulate the symptoms or risk factors of melanoma. Subjects that have melanoma, or at risk for developing melanoma can vary in age, ethnicity, and other parameters. Accordingly, use of the biomarkers disclosed herein, both alone and together in combination with known genetic factors for drug metabolism, allow for a pre-determined level of predictability that a putative therapeutic or prophylactic agent to be tested in a selected subject will be suitable for treating or preventing melanoma in the subject.

To identify therapeutic agents or drugs that are appropriate for a specific subject, a test sample from the subject can also be exposed to a therapeutic agent or a drug, and the level of one or more biomarkers can be determined. The level of one or more biomarkers can be compared to sample derived from the subject before and after subject management for melanoma, e.g., treatment or exposure to a therapeutic agent or a drug, or can be compared to samples derived from one or more subjects who have shown improvements in melanoma risk factors as a result of such treatment or exposure.

Therapeutic agents for treating or reducing the risk of melanoma include, without limitation of the following, radiation therapy with or without therapeutic agents, such as for example, but not limited to, an alkylating agent, an antibiotic agent, an antimetabolic agent, a hormonal agent, a plant-derived agent, an anti-angiogenic agent, a differentiation inducing agent, a cell growth arrest inducing agent, an apoptosis inducing agent, a cytotoxic agent, a biologic agent, a gene therapy agent, a retinoid agent, an HDAC inhibitor, an enzyme inhibitor, a cytokine, a chemokine, an antibody, a DNA molecule, an RNA molecule, a small molecule, a peptide, or a peptidomimetic, or any combination thereof. Specific examples of therapeutic agents include, but are not limited to, fotemustine, dacarbazine, interferon, cisplatin, tamoxifen, interleukin-2, interferon alpha, vinblastin, carmubris, avastin, BRAF-kinase inhibitor, CTLA-4 antibody, angiogenesis inhibitors, targeted immunotherapies, or vaccines.

The term “treating” in its various grammatical forms in relation to the present invention refers to preventing (e.g., chemoprevention), curing, reversing, attenuating, alleviating, minimizing, suppressing or halting the deleterious effects of a disease state, disease progression, disease causative agent (e.g., bacteria or viruses) or other abnormal condition. For example, treatment may involve alleviating a symptom (i.e., not necessary all symptoms) of a disease or attenuating the progression of a disease.

Treatment of cancers such as melanoma, as used herein, refers to partially or totally inhibiting, delaying or preventing the progression of cancer including cancer metastasis; inhibiting, delaying or preventing the recurrence of cancer including cancer metastasis; or preventing the onset or development of cancer (e.g., chemoprevention) in a subject.

As used herein, the term “therapeutically effective amount” is intended to qualify a desired biological response, such as, e.g., is partial or total inhibition, delay or prevention of the progression of cancer including cancer metastasis; inhibition, delay or prevention of the recurrence of cancer including cancer metastasis; or the prevention of the onset or development of cancer (e.g., chemoprevention) in a subject.

### Detecting Biomarkers of the Present Invention

One or more biomarkers disclosed herein can be detected in the practice of the present invention. For example, at least one (1), at least two (2), at least three (3), or all four (4) biomarkers (e.g., each of HELLS, NCAPH, TYMS, and BIRC5) can be detected.

The actual measurement of levels or amounts of the biomarkers can be determined using any method known in the art. These methods include, without limitation, and in particular, PCR methods, including, without limitation, real time PCR, reverse transcriptase PCR and real time reverse transcriptase PCR; sequencing methods, including high-throughput sequencing; nucleic acid chips, mass spectrometry (e.g., laser desorption/ionization mass spectrometry), fluorescence, surface plasmon resonance, ellipsometry and atomic force microscopy. See for example, U.S. Pat. Nos. 5,723,591; 5,801,155 and 6,084,102 and Higuchi, 1992 and 1993. PCR assays may be done, for example, in a multi-well plate formats or in chips, such as the BioTrove OPEN ARRAY Chips (BioTrove, Woburn, Mass.). Preferably, levels of expression of the biomarkers of the present invention are detected by real-time PCR, as described further herein.

For example, sequences within the sequence database entries corresponding to biomarkers of the present invention can be used to construct probes for detecting biomarker RNA sequences in, e.g., Northern blot hybridization analyses or methods which specifically, and, preferably, quantitatively amplify specific nucleic acid sequences. As another example, the sequences can be used to construct primers for specifically amplifying the biomarker sequences in, e.g., amplification-based detection methods such as reverse-transcription based polymerase chain reaction (RT-PCR), preferably quantitative real-time RT-PCR. When alterations in gene expression are associated with gene amplification, deletion, polymorphisms, and mutations, sequence comparisons in test and reference populations can be made by comparing relative amounts of the examined DNA sequences in subject and reference cell populations.

The sample from the subject is typically a biological fluid as described above, and may be the same sample of biological fluid used to conduct the method described above.

The methods for detecting these biomarkers in a sample have many applications. For example, one or more biomarkers can be measured to aid melanoma diagnosis or prognosis. In another example, the methods for detection of the biomarkers can be used to monitor responses in a subject to melanoma treatment. In another example, the methods for detecting biomarkers can be used to assay for and to identify compounds that modulate expression of these biomarkers in vivo or in vitro. In a preferred example, the biomarkers are used to differentiate between the different stages of tumor progression, thus aiding in determining appropriate treatment and extent of metastasis of the tumor.

### Sample Preparation

Nucleic acids may be obtained from the samples in many ways known to one of skill in the art, for example, extraction methods, including e.g., solvent extraction, affinity purification and centrifugation. Selective precipitation can also purify nucleic acids. Chromatography methods may also be utilized including, gel filtration, ion exchange, selective adsorption, or affinity binding. The nucleic acids may be, for example, RNA, DNA or may be synthesized into cDNA. The nucleic acids may be detected using microarray techniques that are well known in the art, for example, Affymetrix arrays followed by multidimensional scaling techniques. [See R. Ekins and F. W. Chu, Microarrays: their origins and applications. Trends Biotechnol., 1999, 17, 217-218; D. D. Shoemaker, et al., Experimental annotation of the human genome using microarray technology, Nature 409(6822): 922-927 (2001) and U.S. Pat. No. 5,750,015.1

In yet another embodiment, a sample can be fractionated using a sequential extraction protocol. In sequential extraction, a sample is exposed to a series of adsorbents to extract different types of biomolecules from a sample. For example, a sample is applied to a first adsorbent to extract certain nucleic acids, and an eluant containing non-adsorbent proteins (i.e., nucleic acids that did not bind to the first adsorbent) is collected. Then, the fraction is exposed to a second adsorbent. This further extracts various nucleic acids from the fraction. This second fraction is then exposed to a third adsorbent, and so on. Any suitable materials and methods can be used to perform sequential extraction of a sample. For example, a series of spin columns comprising different adsorbents can be used. In another example, multi-well plates comprising different adsorbents at its bottom can be used. In another example, sequential extraction can be performed on a probe adapted for use in a gas phase ion spectrometer, wherein the probe surface comprises adsorbents for binding biomolecules. In this embodiment, the sample is applied to a first adsorbent on the probe, which is subsequently washed with an eluant. Biomarkers that do not bind to the first adsorbent are removed with an eluant. The biomarkers that are in the fraction can be applied to a second adsorbent on the probe, and so forth. The advantage of performing sequential extraction on a gas phase ion spectrometer probe is that biomarkers that bind to various adsorbents at every stage of the sequential extraction protocol can be analyzed directly using a gas phase ion spectrometer.

In yet another embodiment, biomolecules in a sample can be separated by high-resolution electrophoresis, e.g., one or two-dimensional gel electrophoresis. A fraction containing a biomarker can be isolated and further analyzed by gas phase ion spectrometry. Preferably, two-dimensional gel electrophoresis is used to generate two-dimensional array of spots of biomolecules, including one or more biomarkers. See, e.g., Jungblut and Thiede, Mass Spectr. Rev. 16: 145-162 (1997). The two-dimensional gel electrophoresis can be performed using methods known in the art. See, e.g., Deutscher (ed.), Methods Enzymol. vol. 182.

In yet another embodiment, high performance liquid chromatography (HPLC) can be used to separate a mixture of biomolecules in a sample based on their different physical properties, such as polarity, charge and size. HPLC instruments typically consist of a reservoir of mobile phase, a pump, an injector, a separation column, and a detector. Biomolecules in a sample are separated by injecting an aliquot of the sample onto the column. Different biomolecules in the mixture pass through the column at different rates due to differences in their partitioning behavior between the mobile liquid phase and the stationary phase. A fraction that corresponds to the molecular weight and/or physical properties of one or more biomarkers can be collected. The fraction can then be analyzed by gas phase ion spectrometry to detect biomarkers.

Optionally, a biomarker can be modified before analysis to improve its resolution or to determine its identity. For example, the biomarkers may be subject to proteolytic digestion before analysis to remove contaminating proteins. Any protease known in the art can be used.

Once captured on a substrate, e.g., biochip, any suitable method, such as those described herein as well as other methods known in the art, can be used to measure a biomarker or biomarkers in a sample.

### Correlation and Data Analysis

The methods for detecting these biomarkers in a sample have many applications. For example, one or more biomarkers can be measured to aid human melanoma diagnosis or prognosis. In another example, the methods for detection of the biomarkers can be used to monitor responses in a subject to melanoma treatment. In other examples, the methods for detecting biomarkers can be used to assay for and to identify compounds that modulate expression of these biomarkers in vivo or in vitro. Differentiation of non-melanoma and melanoma status may be by the detection of one or more of the biomarkers disclosed herein. For example, exemplary biomarkers that may independently discriminate between melanoma statuses include HELLS, NCAPH, TYMS, and BIRC5. Combinations of biomarkers are also useful in the methods of the invention for the determination melanoma and melanoma status. Biomarkers may be detected, determined, and/or monitored in a sample by molecular biological methods, including those disclosed herein, e.g., PCR methods (real-time, reverse transcriptase, PCR).

Detection of biomarkers can be analyzed using any suitable means, including arrays. Nucleic acid arrays may be analyzed using software, for example, Applied Maths, Belgium. GenExplore™: 2-way cluster analysis, principal component analysis, discriminant analysis, self-organizing maps; BioDiscovery, Inc., Los Angeles, Calif. (ImaGene™, special image processing and data extraction software, powered by MatLab®; GeneSight: hierarchical clustering, artificial neural network (SOM), principal component analysis, time series; AutoGene™; CloneTracker™); GeneData AG (Basel, Switzerland); Molecular Pattern Recognition web site at MIT's Whitehead Genome Center; Rosetta Inpharmatics, Kirkland, Wash. Resolver™ Expression Data Analysis System; Scanalytics, Inc., Fairfax, Va. Its MicroArray Suite enables researchers to acquire, visualize, process, and analyze gene expression microarray data; TIGR (The Institute for Genome Research) offers software tools (free for academic institutions) for array analysis. For example, see also Eisen M B, Brown P O., Methods Enzymol. 1999; 303: 179-205.

Detection of biomarkers can be analyzed using any suitable means. In one embodiment, the four-step data reduction algorithm developed by B. Ryu is utilized (Ryu B, Jones J, Blades N.J., Parmigiani G, Hollingsworth M A and Hruban R. Relationships and Differentially Expressed Genes among Pancreatic Cancers examined by Large-scale Serial Analysis of Gene Expression. Cancer Res. 2002; 62:819-826). For example, group comparison was performed using Student's t test. Next, fold differences were evaluated and only those genes with a five-fold or greater expression were kept. The third step involves filtration by sample criteria. However, this step was not applied in this circumstance as expression profiling data from primary melanoma tissue samples was not yet available. In the final step, the genes were further reduced according to the degree of up-regulated expression (Norgauer J, Metzner B, Schraufstatter I. Expression and growth-promoting function of the IL-8 receptor beta in human melanoma cells. J. Immunol. 1996; 156(3): 1132-1137). In one embodiment, data generated, for example, by desorption is analyzed with the use of a programmable digital computer. The computer program generally contains a readable medium that stores codes. Certain code can be devoted to memory that includes the location of each feature on a probe, the identity of the adsorbent at that feature and the elution conditions used to wash the adsorbent. The computer also contains code that receives as input, data on the strength of the signal at various molecular masses received from a particular addressable location on the probe. This data can indicate the number of biomarkers detected, including the strength of the signal generated by each biomarker.

Data analysis can include the steps of determining signal strength (e.g., height of peaks) of a marker detected and removing “outliers” (data deviating from a predetermined statistical distribution). The observed peaks can be normalized, a process whereby the height of each peak relative to some reference is calculated. For example, a reference can be background noise generated by instrument and chemicals (e.g., energy absorbing molecule) which is set as zero in the scale. Then the signal strength detected for each marker or other biomolecules can be displayed in the form of relative intensities in the scale desired (e.g., 100). Alternatively, a standard (e.g., a serum protein) may be admitted with the sample so that a peak from the standard can be used as a reference to calculate relative intensities of the signals observed for each marker or other biomarkers detected.

The computer can transform the resulting data into various formats for displaying. In one format, referred to as “spectrum view or retentate map,” a standard spectral view can be displayed, wherein the view depicts the quantity of marker reaching the detector at each particular molecular weight. In another format, referred to as “peak map,” only the peak height and mass information are retained from the spectrum view, yielding a cleaner image and enabling biomarkers with nearly identical molecular weights to be more easily seen. In yet another format, referred to as “gel view,” each mass from the peak view can be converted into a grayscale image based on the height of each peak, resulting in an appearance similar to bands on electrophoretic gels. In yet another format, referred to as “3-D overlays,” several spectra can be overlaid to study subtle changes in relative peak heights. In yet another format, referred to as “difference map view,” two or more spectra can be compared, conveniently highlighting unique biomarkers and biomarkers which are up- or down-regulated between samples. Biomarker profiles (spectra) from any two samples may be compared visually. In yet another format, Spotfire Scatter Plot can be used, wherein biomarkers that are detected are plotted as a dot in a plot, wherein one axis of the plot represents the apparent molecular of the biomarkers detected and another axis represents the signal intensity of biomarkers detected. For each sample, biomarkers that are detected and the amount of biomarkers present in the sample can be saved in a computer readable medium. This data can then be compared to a control or reference biomarker profile or reference value (e.g., a profile or quantity of biomarkers detected in control, e.g., subjects in whom human melanoma is undetectable).

When the sample is measured and data is generated, e.g., by mass spectrometry, the data is then analyzed by a computer software program. Generally, the software can comprise code that converts signal from the mass spectrometer into computer readable form. The software also can include code that applies an algorithm to the analysis of the signal to determine whether the signal represents a “peak” in the signal corresponding to a marker of this invention, or other useful biomarkers. The software also can include code that executes an algorithm that compares signal from a test sample to a typical signal characteristic of “normal” and melanoma and determines the closeness of fit between the two signals. The software also can include code indicating which the test sample is closest, thereby providing a probable diagnosis.

In preferred methods of the present invention, multiple biomarkers are measured. The use of multiple biomarkers increases the predictive value of the test and provides greater utility in diagnosis, toxicology, subject stratification and subject monitoring. The process called “Pattern recognition” detects the patterns formed by multiple biomarkers greatly improves the sensitivity and specificity of clinical proteomics for predictive medicine. Subtle variations in data from clinical samples indicate that certain patterns of protein expression can predict phenotypes such as the presence or absence of a certain disease, a particular stage of melanoma progression, or a positive or adverse response to drug treatments.

Classification models can be formed using any suitable statistical classification (or “learning”) method that attempts to segregate bodies of data into classes based on objective parameters present in the data. Classification methods may be either supervised or unsupervised. Examples of supervised and unsupervised classification processes are described in Jain, “Statistical Pattern Recognition: A Review”, IEEE Transactions on Pattern Analysis and Machine Intelligence, Vol. 22, No. 1, January 2000, which is herein incorporated by reference in its entirety. In supervised classification, training data containing examples of known categories are presented to a learning mechanism, which learns one more sets of relationships that define each of the known classes. New data may then be applied to the learning mechanism, which then classifies the new data using the learned relationships. Examples of supervised classification processes include linear regression processes (e.g., multiple linear regression (MLR), partial least squares (PLS) regression and principal components regression (PCR)), binary decision trees (e.g., recursive partitioning processes such as CART—classification and regression trees), artificial neural networks such as back propagation networks, discriminant analyses (e.g., Bayesian classifier or Fischer analysis), logistic classifiers, and support vector classifiers (support vector machines). A preferred supervised classification method is a recursive partitioning process.

Recursive partitioning processes use recursive partitioning trees to classify spectra derived from unknown samples. In other embodiments, the classification models that are created can be formed using unsupervised learning methods. Unsupervised classification attempts to learn classifications based on similarities in the training data set, without pre classifying the spectra from which the training data set was derived. Unsupervised learning methods include cluster analyses. A cluster analysis attempts to divide the data into “clusters” or groups that ideally should have members that are very similar to each other, and very dissimilar to members of other clusters. Similarity is then measured using some distance metric, which measures the distance between data items, and clusters together data items that are closer to each other. Clustering techniques include the MacQueen's K-means algorithm and the Kohonen's Self-Organizing Map algorithm.

Learning algorithms asserted for use in classifying biological information are described in, for example, International Application No. WO 01/31580 (Barnhill et al., “Methods and devices for identifying patterns in biological systems and methods of use thereof,” May 3, 2001); U.S. Patent Application No. 2002/0193950 A1 (Gavin et al., “Method or analyzing mass spectra,” Dec. 19, 2002); U.S. Patent Application No. 2003/0004402 A1 (Hitt et al., “Process for discriminating between biological states based on hidden patterns from biological data,” Jan. 2, 2003); and U.S. Patent Application No. 2003/0055615 A1 (Zhang and Zhang, “Systems and methods for processing biological expression data” Mar. 20, 2003).

More specifically, to obtain the biomarkers the peak intensity data of samples from subjects, e.g., melanoma subjects, and healthy controls are used as a “discovery set.” These data were combined and randomly divided into a training set and a test set to construct and test multivariate predictive models using a non-linear version of Unified Maximum Separability Analysis (“USMA”) classifiers. Details of USMA classifiers are described in U.S. Patent Application No. 2003/0055615. The invention provides methods for aiding a human melanoma diagnosis using one or more biomarkers as specified herein. In particular, HELLS, NCAPH, TYMS, and BIRC5, particularly HELLS and NCAPH, are useful biomarkers for more aggressive melanoma. These biomarkers can be used alone, in combination with other biomarkers in any set, or with entirely different biomarkers in aiding human melanoma diagnosis. The biomarkers are differentially present in samples of a human melanoma subject and a normal subject in whom human melanoma is undetectable. For example, the biomarkers of the current invention are expressed at an elevated level and/or are present at a higher frequency in human melanoma subjects when compared with normal subjects. Therefore, detection of one or more of these biomarkers in a person would provide useful information regarding the probability that the person may have melanoma.

In any of the methods disclosed herein, the data from the sample may be fed directly from the detection means into a computer containing the diagnostic algorithm. Alternatively, the data obtained can be fed manually, or via an automated means, into a separate computer that contains the diagnostic algorithm. Accordingly, embodiments of the invention include methods involving correlating the detection of the biomarker or biomarkers with a probable diagnosis of melanoma. The correlation may take into account the amount of the biomarker or biomarkers in the sample compared to a control amount of the biomarker or biomarkers (up or down regulation of the biomarker or biomarkers) (e.g., in normal subjects in whom melanoma is undetectable). The correlation may take into account the presence or absence of the biomarkers in a test sample and the frequency of detection of the same biomarkers in a control. The correlation may take into account both of such factors to facilitate determination of whether a subject has a melanoma or not.

In a preferred embodiment, any one of the biomarkers HELLS, NCAPH, TYMS, and BIRC5, or a combination thereof, are used to make a correlation with melanoma, wherein the melanoma may be any subtype, e.g., superficial spreading, nodular, acrolentiginous, and lentigo maligna. In other embodiments, the measurement of biomarkers can involve quantifying the biomarkers to correlate the detection of biomarkers with a probable diagnosis of melanoma. Thus, if the amount of the biomarkers detected in a subject being tested is elevated compared to a control amount, then the subject being tested has a higher probability of having melanoma.

The correlation may take into account the amount of the biomarker or biomarkers in the sample compared to a control amount of the biomarker or biomarkers (up or down regulation of the biomarker or biomarkers) (e.g., in normal subjects or in non-melanoma subjects such as where melanoma is undetectable). A control can be, e.g., the average or median amount of biomarker present in comparable samples of normal subjects in normal subjects or in non-melanoma subjects such as where melanoma is undetectable. The control amount is measured under the same or substantially similar experimental conditions as in measuring the test amount. The correlation may take into account the presence or absence of the biomarkers in a test sample and the frequency of detection of the same biomarkers in a control. The correlation may take into account both of such factors to facilitate determination of melanoma status.

In certain embodiments of the methods of qualifying melanoma status, the methods further comprise managing subject treatment based on the status. As before the management of the subject describes the actions of the physician or clinician subsequent to determining melanoma status. For example, if the result of the methods of the present invention is inconclusive or there is reason that confirmation of status is necessary, the physician may order more tests (e.g., CT scans, PET scans, MRI scans, PET-CT scans, X-rays, biopsies, blood tests (LFTs, LDH). Alternatively, if the status indicates that treatment is appropriate, the physician may schedule the subject for treatment. In other instances, the subject may receive therapeutic treatments, either in lieu of or in addition to, surgery. No further action may be warranted. Furthermore, if the results show that treatment has been successful, a maintenance therapy or no further management may be necessary. Therapeutic agents may include, one or more of fotemustine, dacarbazine, interferon, cisplatin, tamoxifen, interleukin-2, interferon alpha, vinblastin, or carmubris, but are not limited to these examples.

The invention also provides for such methods where the biomarkers (or specific combination of biomarkers) are measured again after subject management. In these cases, the methods are used to monitor the status of the melanoma, e.g., response to melanoma treatment, remission of the disease or progression of the disease. Because of the ease of use of the methods and the lack of invasiveness of the methods, the methods can be repeated after each treatment the subject receives. This allows the physician to follow the effectiveness of the course of treatment. If the results show that the treatment is not effective, the course of treatment can be altered accordingly. This enables the physician to be flexible in the treatment options.

In another example, the methods for detecting biomarkers can be used to assay for and to identify compounds that modulate expression or activity of these biomarkers in vivo or in vitro.

The methods of the present invention have other applications as well. For example, the biomarkers can be used to screen for compounds that modulate the expression of the biomarkers in vitro or in vivo, which compounds in turn may be useful in treating or preventing melanoma in subjects. In another example, the biomarkers can be used to monitor the response to treatments for melanoma. In yet another example, the biomarkers can be used in heredity studies to determine if the subject is at risk for developing melanoma. For instance, certain biomarkers may be genetically linked. This can be determined by, e.g., analyzing samples from a population of melanoma subjects whose families have a history of melanoma. The results can then be compared with data obtained from, e.g., melanoma subjects whose families do not have a history of melanoma. The biomarkers that are genetically linked may be used as a tool to determine if a subject whose family has a history of melanoma is pre-disposed to having melanoma. In a preferred embodiment of the invention, a diagnosis based on the presence or absence in a test subject of any the biomarkers of this invention is communicated to the subject as soon as possible after the diagnosis is obtained. The diagnosis may be communicated to the subject by the subject's treating physician. Alternatively, the diagnosis may be sent to a test subject by email or communicated to the subject by phone. A computer may be used to communicate the diagnosis by email or phone. In certain embodiments, the message containing results of a diagnostic test may be generated and delivered automatically to the subject using a combination of computer hardware and software which will be familiar to artisans skilled in telecommunications. One example of a healthcare-oriented communications system is described in U.S. Pat. No. 6,283,761; however, the present invention is not limited to methods which utilize this particular communications system. In certain embodiments of the methods of the invention, all or some of the method steps, including the assaying of samples, diagnosing of diseases, and communicating of assay results or diagnoses, may be carried out in diverse (e.g., foreign) jurisdictions.

Methods of the invention for determining the melanoma status of a subject, include for example, obtaining a biomarker profile from a sample taken from the subject; and comparing the subject's biomarker profile to a reference biomarker profile obtained from a reference population, wherein the comparison is capable of classifying the subject as belonging to or not belonging to the reference population; wherein the subject's biomarker profile and the reference biomarker profile comprise one or more biomarkers as described herein.

The method may further comprise repeating the method at least once, wherein the subject's biomarker profile is obtained from a separate sample taken each time the method is repeated. Samples from the subject may be taken at any time, for example, the samples may be taken 24 hours apart or any other time determined useful.

Such comparisons of the biomarker profiles can determine melanoma status in the subject with an accuracy of at least about 60%, 70%, 80%, 90%, 95%, and approaching 100%. The reference biomarker profile can be obtained from a population comprising a single subject, at least two subjects, at least 20 subjects or more. The number of subjects will depend, in part, on the number of available subjects, and the power of the statistical analysis necessary.

A dataset can be analyzed by multiple classification algorithms. Some classification algorithms provide discrete rules for classification; others provide probability estimates of a certain outcome (class). In the latter case, the decision (diagnosis) is made based on the class with the highest probability. For example, consider the three-class problem: healthy, benign, and melanoma. Suppose that a classification algorithm (e.g. nearest neighbor) is constructed and applied to sample A, and the probability of the sample being healthy is 0, benign is 33%, and melanoma is 67%. Sample A would be diagnosed as being melanoma. This approach, however, does not take into account any “fuzziness” in the diagnosis, e.g., that there was a certain probability that the sample was benign. Therefore, the diagnosis would be the same as for sample B, which has a probability of 0 of being healthy or benign and a probability of 1 of being melanoma. Other classification algorithms and formulae include, but are not limited to, Principal Component Analysis (PCA), cross-correlation, factor rotation, Logistic Regression (LogReg), Linear Discriminant Analysis (LDA), Eigengene Linear Discriminant Analysis (ELDA), Support Vector Machines (SVM), Random Forest (RF), Recursive Partitioning Tree (RPART), as well as other related decision tree classification techniques, Shrunken Centroids (SC), StepAIC, Kth-Nearest Neighbor, Boosting, Decision Trees, Neural Networks, Bayesian Networks, Support Vector Machines, Leave-One-Out (LOO), 10-Fold cross-validation (10-Fold CV), and Hidden Markov Models, among others.

### Antibodies

As used herein, the term “antibody” means not only intact antibody molecules, but also fragments of antibody molecules that retain immunogen binding ability. Such fragments are also well known in the art and are regularly employed both in vitro and in vivo. Accordingly, as used herein, the term “antibody” means not only intact immunoglobulin molecules but also the well-known active fragments F(ab′)2, and Fab. F(ab′)2, and Fab fragments which lack the Fe fragment of intact antibody, clear more rapidly from the circulation, and may have less non-specific tissue binding of an intact antibody (Wahl et al., (1983) J. Nucl. Med. 24:316-325. The antibodies of the invention comprise whole native antibodies, bispecific antibodies; chimeric antibodies; Fab, Fab′, single chain V region fragments (scFv) and fusion polypeptides.

“Humanized” antibodies are antibodies in which at least part of the sequence has been altered from its initial form to render it more like human immunoglobulins. Techniques to humanize antibodies are particularly useful when non-human animal (e.g., murine) antibodies are generated. Examples of methods for humanizing a murine antibody are provided in U.S. Pat. Nos. 4,816,567, 5,530,101, 5,225,539, 5,585,089, 5,693,762 and 5,859,205.

### Inhibitory Nucleic Acids

The invention encompasses the use of inhibitory nucleic acids. Inhibitory nucleic acids may be designed based on identification of biomarkers that indicate melanoma status and progression of disease in a subject. In certain preferred examples, the invention features HELLS, NCAPH, TYMS, and BIRC5 inhibitory nucleic acid molecules. HELLS, NCAPH, TYMS, and BIRC5 inhibitory nucleic acid molecules are essentially nucleobase oligomers that may be employed as single-stranded or double-stranded nucleic acid molecule to decrease HELLS, NCAPH, TYMS, and BIRC5 expression.

In one approach, the HELLS, NCAPH, TYMS, or BIRC5 inhibitory nucleic acid molecule is a double-stranded RNA used for RNA interference (RNAi)-mediated knock-down of HELLS, NCAPH, TYMS, and BIRC5 gene expression. In one embodiment, a double-stranded RNA (dsRNA) molecule is made that includes between eight and twenty-five (e.g., 8, 10, 12, 15, 16, 17, 18, 19, 20, 21, 22, 23, 24, 25) consecutive nucleobases of a nucleobase oligomer of the invention. The dsRNA can be two complementary strands of RNA that have duplexed, or a single RNA strand that has self-duplexed (small hairpin (sh)RNA). Typically, dsRNAs are about 21 or 22 base pairs, but may be shorter or longer (up to about 29 nucleobases) if desired. Double stranded RNA can be made using standard techniques (e.g., chemical synthesis or in vitro transcription). Kits are available, for example, from Ambion (Austin, Tex.) and Epicentre (Madison, Wis.). Methods for expressing dsRNA in mammalian cells are described in Brummelkamp et al. (2002) Science 296:550-553; Paddison et al. (2002) Genes Dev. 16:948-958; Paul et al. (2002) Nature Biotechnol. 20:505-508; Sui et al. (2002) Proc. Natl. Acad. Sci. USA 99:5515-5520; Yu et al. (2002) Proc. Natl. Acad. Sci. USA 99:6047-6052; Miyagishi et al. (2002) Nature Biotechnol. 20:497-500; and Lee et al. (2002) Nature Biotechnol. 20:500-505, each of which is hereby incorporated by reference.

An inhibitory nucleic acid molecule that “corresponds” to one of HELLS, NCAPH, TYMS, or BIRC5 gene comprises at least a fragment of the double-stranded gene, such that each strand of the double-stranded inhibitory nucleic acid molecule is capable of binding to the complementary strand of a target HELLS, NCAPH, TYMS, or BIRC5 gene. The inhibitory nucleic acid molecule need not have perfect correspondence to the reference HELLS, NCAPH, TYMS, or BIRC5 sequence. In one embodiment, an siRNA has at least about 85%, 90%, 95%, 96%, 97%, 98%, or even 99% sequence identity with the target nucleic acid. For example, a 19 base pair duplex having 1-2 base pair mismatch is considered useful in the methods of the invention. In other embodiments, the nucleobase sequence of the inhibitory nucleic acid molecule exhibits 1, 2, 3, 4, 5 or more mismatches. The inhibitory nucleic acid molecules provided by the invention are not limited to siRNAs, but include any nucleic acid molecule sufficient to decrease the expression of a HELLS, NCAPH, TYMS, or BIRC5 nucleic acid molecule or polypeptide. Each of the DNA sequences provided herein may be used, for example, in the discovery and development of therapeutic antisense nucleic acid molecule to decrease the expression of any one of HELLS, NCAPH, TYMS, or BIRC5.

The invention further provides catalytic RNA molecules or ribozymes. Such catalytic RNA molecules can be used to inhibit expression of a HELLS, NCAPH, TYMS, or BIRC5 nucleic acid molecule in vivo. The inclusion of ribozyme sequences within an antisense RNA confers RNA-cleaving activity upon the molecule, thereby increasing the activity of the constructs. The design and use of target RNA-specific ribozymes is described in Haseloff et al., (1988) Nature 334:585-591, and U.S. Patent Application No. 2003/0003469, each of which is incorporated by reference. In various embodiments of this invention, the catalytic nucleic acid molecule is formed in a hammerhead or hairpin motif. Examples of such hammerhead motifs are described by Rossi et al., (1992) AIDS Research and Human Retroviruses, 8:183. Example of hairpin motifs are described by Hampel et al., “RNA Catalyst for Cleaving Specific RNA Sequences,” filed Sep. 20, 1989 (U.S. patent application Ser. No. 07/409,666), which is a continuation-in-part of U.S. patent application Ser. No. 07/247,100 filed Sep. 20, 1988; Hampel and Tritz, (1989) Biochemistry, 28:4929, and Hampel et al., (1990) Nucl. Acids Res., 18: 299. These specific motifs are not limiting in the invention and those skilled in the art will recognize that all that is important in an enzymatic nucleic acid molecule of this invention is that it has a specific substrate binding site which is complementary to one or more of the target gene RNA regions, and that it have nucleotide sequences within or surrounding that substrate binding site which impart an RNA cleaving activity to the molecule. After a subject is diagnosed as having melanoma, or at risk for recurrence of melanoma, a method of treatment is selected.

In one embodiment, the inhibitory nucleic acid molecules of the invention are administered systemically in dosages between about 1 and 100 mg/kg (e.g., 1, 5, 10, 20, 25, 50, 75, and 100 mg/kg). In other embodiments, the dosage ranges from between about 25 and 500 mg/m²/day. Desirably, a human patient having melanoma receives a dosage between about 50 and 300 mg/m²/day (e.g., 50, 75, 100, 125, 150, 175, 200, 250, 275, and 300).

A desirable inhibitory nucleic acid molecule is one based on 2′-modified oligonucleotides containing oligodeoxynucleotide gaps with some or all internucleotide linkages modified to phosphorothioates for nuclease resistance. The presence of methylphosphonate modifications increases the affinity of the oligonucleotide for its target RNA and thus reduces the IC₅₀. This modification also increases the nuclease resistance of the modified oligonucleotide. It is understood that the methods and reagents of the present invention may be used in conjunction with any technologies that may be developed to enhance the stability or efficacy of an inhibitory nucleic acid molecule.

Inhibitory nucleic acid molecules include nucleobase oligomers containing modified backbones or non-natural internucleoside linkages. Oligomers having modified backbones include those that retain a phosphorus atom in the backbone and those that do not have a phosphorus atom in the backbone. For the purposes of this specification, modified oligonucleotides that do not have a phosphorus atom in their internucleoside backbone are also considered to be nucleobase oligomers. Nucleobase oligomers that have modified oligonucleotide backbones include, for example, phosphorothioates, chiral phosphorothioates, phosphorodithioates, phosphotriesters, aminoalkyl-phosphotriesters, methyl and other alkyl phosphonates including 3′-alkylene phosphonates and chiral phosphonates, phosphinates, phosphoramidates, thionophosphoramidates, thionoalkylphosphonates, thionoalkylphosphotriesters, and boranophosphates. Various salts, mixed salts and free acid forms are also included. Representative United States patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, each of which is herein incorporated by reference

Nucleobase oligomers having modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. Representative United States patents that teach the preparation of the above oligonucleotides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

Nucleobase oligomers may also contain one or more substituted sugar moieties. Such modifications include 2′-O-methyl and 2′-methoxyethoxy modifications. Another desirable modification is 2′-dimethylaminooxyethoxy, 2′-aminopropoxy and 2′-fluoro. Similar modifications may also be made at other positions on an oligonucleotide or other nucleobase oligomer, particularly the 3′ position of the sugar on the 3′ terminal nucleotide. Nucleobase oligomers may also have sugar mimetics such as cyclobutyl moieties in place of the pentofuranosyl sugar. Representative United States patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, each of which is herein incorporated by reference in its entirety. In other nucleobase oligomers, both the sugar and the internucleoside linkage, i.e., the backbone, are replaced with novel groups. The nucleobase units are maintained for hybridization with an HELLS, NCAPH, TYMS, or BIRC5 nucleic acid molecule. Methods for making and using these nucleobase oligomers are described, for example, in “Peptide Nucleic Acids (PNA): Protocols and Applications” Ed. P. E. Nielsen, Horizon Press, Norfolk, United Kingdom, 1999. Representative United States patents that teach the preparation of PNAs include, but are not limited to, U.S. Pat. Nos. 5,539,082; 5,714,331; and 5,719,262, each of which is herein incorporated by reference. Further teaching of PNA compounds can be found in Nielsen et al, (1991) Science 254: 1497-1500.

### Biomarkers and Methods of the Invention

In one aspect, the invention also includes melanoma candidate genes, which are useful as therapeutic targets. These genes include, for example, those listed herein, e.g. biomarkers identified as HELLS, NCAPH, TYMS, and BIRC5.

In particular, as described, HELLS and NCAPH represent useful therapeutic targets.

The methods of the present invention have other applications as well. For example, the biomarkers can be used to screen for compounds that modulate the expression of the biomarkers in vitro or in vivo, which compounds in turn may be useful in treating or preventing melanoma in subjects. In another example, the biomarkers can be used to monitor the response to treatments for melanoma. In yet another example, the biomarkers can be used in heredity studies to determine if the subject is at risk for developing melanoma. Thus, for example, the kits of this invention could include a solid substrate having a hydrophobic function, such as a protein biochip (e.g., a Ciphergen ProteinChip array), to detect the product of the nucleic acid biomarkers, and a buffer for washing the substrate, as well as instructions providing a protocol to measure the biomarkers of this invention on the chip and to use these measurements to diagnose melanoma. Methods for identifying a candidate compound for treating melanoma may comprise, for example, contacting one or more of the protein products of the biomarkers of the invention with a test compound; and determining whether the test compound interacts with the protein, wherein a compound that interacts with the protein is identified as a candidate compound for treating melanoma. Compounds suitable for therapeutic testing may be screened initially by identifying compounds which interact with one or more of the proteins that are the products of the biomarkers identified herein. By way of example, screening might include recombinantly expressing a protein, purifying the protein, and affixing the protein to a substrate. Test compounds would then be contacted with the substrate, typically in aqueous conditions, and interactions between the test compound and the protein are measured, for example, by measuring elution rates as a function of salt concentration. Certain proteins may recognize and cleave one or more proteins of this invention, in which case the proteins may be detected by monitoring the digestion of one or more proteins in a standard assay, e.g., by gel electrophoresis of the proteins.

In a related embodiment, the ability of a test compound to inhibit the activity of one or more of the proteins of this invention may be measured. One of skill in the art will recognize that the techniques used to measure the activity of a particular protein will vary depending on the function and properties of the protein. For example, an enzymatic activity of a protein may be assayed provided that an appropriate substrate is available and provided that the concentration of the substrate or the appearance of the reaction product is readily measurable. The ability of potentially therapeutic test compounds to inhibit or enhance the activity of a given protein may be determined by measuring the rates of catalysis in the presence or absence of the test compounds. The ability of a test compound to interfere with a non-enzymatic (e.g., structural) function or activity of one of the protein of this invention may also be measured. For example, the self-assembly of a multi-protein complex which includes one of the proteins of this invention may be monitored by spectroscopy in the presence or absence of a test compound. Alternatively, if the protein is a non-enzymatic enhancer of transcription, test compounds which interfere with the ability of the protein to enhance transcription may be identified by measuring the levels of protein-dependent transcription in vivo or in vitro in the presence and absence of the test compound.

Test compounds capable of modulating the activity of any of the proteins may be administered to subjects who are suffering from or are at risk of developing melanoma. For example, the administration of a test compound which decreases the activity of a particular protein may decrease the risk from melanoma in a subject if the increased activity of the protein is responsible, at least in part, for the onset of melanoma or for the aggressive state of a melanoma.

In a related embodiment, the ability of a test compound to inhibit the gene expression of one or more of the biomarkers of this invention may be measured. One of skill in the art will recognize that the techniques used to measure the levels of a particular can be applied to a sample of melanoma cells and test compounds can be evaluated for the ability to reduce the level of expression of the biomarker, thus rendering the melanoma less aggressive.

At the clinical level, screening a test compound includes obtaining samples from test subjects before and after the subjects have been exposed to a test compound. The CNA levels in the samples of one or more of the biomarkers of this invention may be measured and analyzed to determine whether the levels of the biomarkers change after exposure to a test compound. The samples may be analyzed by PCR, as described herein, or the samples may be analyzed by any appropriate means known to one of skill in the art. In a further embodiment, the changes in the level of expression of one or more of the biomarkers may be measured using in vitro methods and materials. For example, human cultured cells which express, or are capable of expressing, one or more of the biomarkers of this invention may be contacted with test compounds. Subjects who have been treated with test compounds will be routinely examined for any physiological effects which may result from the treatment. As one embodiment, the test compounds will be evaluated for their ability to decrease disease likelihood in a subject. Alternatively, if the test compounds are administered to subjects who have previously been diagnosed with melanoma, test compounds will be screened for their ability to slow or stop the progression of the disease.

Methods of identifying therapeutic targets for melanoma generally comprise comparing an expression profile of a melanoma cell with an expression profile of a reference cell, wherein the comparison is capable of classifying proteins or transcripts in the profile as being associated with melanoma proliferation and/or metastasis. Methods for evaluation of proliferation and/or metastasis are well known to one of skill in the art. Reference cells may be normal cells (cells that are not melanoma cells). The reference cells may be primary cultured cells, fresh blood cells, established cell lines or other cells determined to be appropriate to one of skill in the art. Transcripts and proteins associated with melanoma metastasis include gene products that differentiate between normal and melanoma cell lines and, alternatively, that differentiate among non-metastatic versus metastatic melanomas. The transcripts and proteins may also differentiate between melanoma and other forms of cancer. The proteins may be secreted proteins, such that they are easily detectable from a blood sample.

### Kits

The invention provides kits for qualifying melanoma status and/or detecting or diagnosing melanoma, wherein the kits can be used to detect the biomarkers of the present invention. For example, the kits can be used to detect any one or more of the biomarkers described herein, which biomarkers are differentially present in samples of melanoma subjects and normal subjects. The kits of the invention have many applications. For example, the kits can be used in any one of the methods of the invention described herein, such as, inter alia, to differentiate if a subject has melanoma or has a negative diagnosis, thus aiding a melanoma diagnosis. In another example, the kits can be used to identify compounds that modulate expression of one or more of the biomarkers in in vitro or in vivo animal models for melanoma.

Generally, kits of the present invention include a biomarker-detection reagent, e.g., nucleic acids that specifically identify one or more biomarker nucleic acids by having homologous nucleic acid sequences, such as oligonucleotide sequences complementary to a portion of the biomarker nucleic acids. The oligonucleotides can be fragments of the biomarker genes. The oligonucleotides may be single stranded or double stranded. For example the oligonucleotides can be 200, 150, 100, 50, 25, 10 or less nucleotides in length. The kit may contain in separate containers a nucleic acid (either already bound to a solid matrix or packaged separately with reagents for binding them to the matrix), control formulations (positive and/or negative), and/or a detectable label such as fluorescein, green fluorescent protein, rhodamine, cyanine dyes, Alexa dyes, luciferase, radiolabels, among others. Instructions (e.g., written, tape, VCR, CD-ROM, etc.) for carrying out the assay and for correlation to melanoma status may be included in the kit.

For example, biomarker detection reagents can be immobilized on a solid matrix such as a porous strip to form at least one biomarker detection site. The measurement or detection region of the porous strip may include a plurality of sites containing a nucleic acid. A test strip may also contain sites for negative and/or positive controls. Alternatively, control sites can be located on a separate strip from the test strip. Optionally, the different detection sites may contain different amounts of immobilized nucleic acids, e.g., a higher amount in the first detection site and lesser amounts in subsequent sites. Upon the addition of test sample, the number of sites displaying a detectable signal provides a quantitative indication of the amount of biomarkers present in the sample. The detection sites may be configured in any suitably detectable shape and are typically in the shape of a bar or dot spanning the width of a test strip.

Alternatively, the kit contains a nucleic acid substrate array comprising one or more nucleic acid sequences, e.g., primers for nucleic acid amplification. The nucleic acids on the array specifically identify one or more nucleic acid sequences represented by the biomarkers of the present invention. In various embodiments, the expression of 2, 3, or all 4 of the sequences represented by the biomarkers described herein can be identified by virtue of binding to the array. The substrate array can be on, e.g., a solid substrate, e.g., a “chip” as described in U.S. Pat. No. 5,744,305. Alternatively, the substrate array can be a solution array, e.g., xMAP (Luminex, Austin, Tex.), Cyvera (Illumina, San Diego, Calif.), CellCard (Vitra Bioscience, Mountain View, Calif.) and Quantum Dots' Mosaic (Invitrogen, Carlsbad, Calif.). The kit may also contain reagents, and/or enzymes for amplifying or isolating sample DNA. The kits may include reagents for real-time PCR, for example, TaqMan probes and/or primers, and enzymes.

In one embodiment, a kit comprises: (a) a substrate comprising an adsorbent thereon, wherein the adsorbent retains or is otherwise suitable for binding a biomarker, and (b) instructions to detect the biomarker or biomarkers by contacting a sample with the adsorbent and detecting the biomarker or biomarkers retained by the adsorbent. In some embodiments, the kit may comprise an eluant (as an alternative or in combination with instructions) or instructions for making an eluant, wherein the combination of the adsorbent and the eluant allows detection of the biomarkers using gas phase ion spectrometry. Such kits can be prepared from the materials described above, and the previous discussion of these materials (e.g., probe substrates, adsorbents, washing solutions, etc.) is fully applicable to this section and will not be repeated.

In another embodiment, the kit may comprise a first substrate comprising an adsorbent thereon (e.g., a particle functionalized with an adsorbent) and a second substrate onto which the first substrate can be positioned to form a probe, which is removably insertable into a gas phase ion spectrometer. In other embodiments, the kit may comprise a single substrate, which is in the form of a removably insertable probe with adsorbents on the substrate. In yet another embodiment, the kit may further comprise a pre-fractionation spin column (e.g., Cibacron blue agarose column, anti-HSA agarose column, K-30 size exclusion column, Q-anion exchange spin column, single stranded DNA column, lectin column, etc.).

Optionally, the kit may further comprise pre-fractionation spin columns. In some embodiments, the kit may further comprise instructions for suitable operation parameters in the form of a label or a separate insert. Optionally, the kit may further comprise a standard or control information so that the test sample can be compared with the control information standard to determine if the test amount of a biomarker detected in a sample is a diagnostic amount consistent with a diagnosis of melanoma.

This invention is further illustrated by the following examples, which should not be construed as limiting. All documents mentioned herein are incorporated herein by reference.

### EXAMPLES

### Example 1

### Measurement of Metastasis-Associated Gene Transcript Levels in Melanoma Patients

HELLS gene encodes a lymphoid-specific helicase. HELLS is a member of a SWI/SNF2 subfamily of helicases which is critical in the regulation of chromatin remodeling, DNA replication, repair, recombination, methylation, and transcription [Sun, L. Q., and Arceci, R. J. (2005). Altered epigenetic patterning leading to replicative senescence and reduced longevity. A role of a novel SNF2 factor, PASG. Cell Cycle 4, 3-5]. The molecular function of HELLS is de novo DNA methylation which is an important player in embryonic stem cell differentiation by silencing sternness genes [Xi, S., Geiman, T. M., Briones, V., Guang Tao, Y., Xu, H., and Muegge, K. (2009). Lsh participates in DNA methylation and silencing of stem cell genes. Stem Cells (Dayton, Ohio) 27, 2691-702]. However, functional roles of HELLS protein in tumor initiation and metastasis development would not a priori have been predicted to be a prognostic biomarker. Higher levels of circulating HELLS mRNA are a reflection of higher numbers of circulating tumor cells in metastatic patient blood, implicating HELLS in tumor cell migration and invasion, because metastasis-competent cells are most likely invasive and migratory, enabling access to a tumor vascular system. Although HELLS mRNA expression levels are very low in normal peripheral blood lymphocytes, when such lymphocytes are activated and stimulated to proliferate, expression levels are significantly increased [Lee, D. W., Zhang, K., Ning, Z. Q., Raabe, E. H., Tintner, S., Wieland, R., Wilkins, B. J., Kim, J. M., Blough, R. I., and Arceci, R. J. (2000). Proliferation-associated SNF2-like gene (PASG): a SNF2 family member altered in leukemia. Cancer Res. 60, 3612-22]. This finding indicates that expression levels of HELLS are useful as a biomarker of metastasis in subjects.

### Materials and Methods

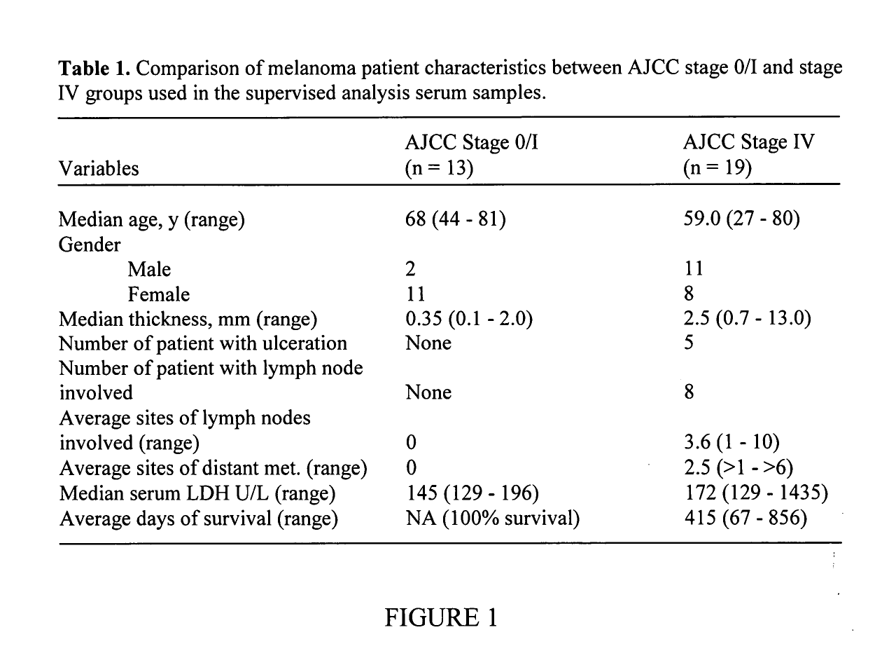

Patient Populations. Melanoma patients with available fresh frozen serum samples in the Nevada Cancer Institute (NVCI) tissue bank linked with characterization of patient's clinical stage and risk factors were identified from the files of the Laboratory Core. Serum samples from 32 cutaneous melanoma patients evaluated at NVCI between May 2006 and December 2009 were used. These samples included 13 samples from Stage 0/I patients with localized primary melanoma and 19 samples from Stage IV patients with distant organ melanoma. Blood samples were obtained at the time of initial diagnosis (Stage 0/I) and detection of metastasis (stage IV). Twenty serum samples from sex- and age-matched healthy controls with no history of illness (average age, 57.8 yr) were used as a normal control. Key clinical and histopathologic characteristics of the melanoma patients included in this study are summarized in FIG. 1. This analysis was approved by the University Medical Center IRB, Las Vegas, Nev.

RNA Extraction and Reverse Transcription. In vitro transcribed luciferase mRNA (10 ng) was spiked into 250 μl serum samples to monitor quality of subsequent RNA extraction and reverse transcription steps. Total RNA was isolated from each serum sample using a mirvanaPARIS Kit (Applied Biosystems/Ambion, Austin, Tex.) according to the manufacturer's instruction. First-strand cDNA was generated using High Capacity RNA-to-cDNA Kit (Applied Biosystems, Foster City, Calif.) using random hexamers according to the manufacturer's protocol.

Duplex Quantitative Real-time PCR. Primers and TaqMan Probes for corresponding genes were selected from the mRNA sequences obtained from NCBI's reference sequence database using Primer Expression Software 3.0 (Applied Biosystems, Foster City, Calif.). The primer sets and TaqMan probes for duplex quantitative real-time PCR were follows: HELLS, 5′-CCCTCCTTTCTTCTAGTAATGCAGTT-3′ (forward), 5′-CCCAATCTCTCCCCATGAAAA-3′ (reverse), and 6FAM-ATGGGCTTTAGGTACTTC-BHQ-1 (probe); TYMS, 5′-TTTTGGACAGCCTGGGATTC-3′ (forward), 5′-GCCATAAACTGGGCCCAAGT-3′ (reverse), and 6FAM-CCACCAGAGAAGAAG-BHQ-1 (probe); NCAPH, 5′-CTGGATTACAGGCTGCTGAC-3′ (forward), 5′-GGTCAGAGTTCCCAACAGGT-3′ (reverse), and 6FAM-CACAAATAAGTCATCCAAAT-BHQ-[(probe); BIRC5,5′-CGCTTTCCTTTCTGTCAAGA-3′ (forward), 5′-CTTGGCTCTTTCTCTGTCCA-3′ (reverse), and 6FAM-AGTTTGAAGAATTAACCCTT-BHQ-1 (probe); ACTB, 5′-CTGGAACGGTGAAGGTGACA-3′ (forward), 5′-CGGCCACATTGTGAACTTTG-3′ (reverse), and 6FAM-TCGGTTGGAGCGAGC-BHQ-1 (probe); luciferase, 5′-CGTACGTGATGTTCACCTCGAT-3′ (forward), 5′-CGCCCTGGTTCCTGGAA-3′ (reverse), and VIC-TGCATCTGTAAAAGCA-BHQ-1 (probe).

Two different types of fluorescent dye labeled probes were utilized for the duplex quantitative real-time PCR assay. This allows detection and quantitation of levels of two gene transcripts in a single reaction mixture. PCR amplifications were performed using an ABI 7500 Fast Real-Time PCR System (Applied Biosystems, Foster City, Calif.) and were performed in a reaction volume of 20 μl containing 1× TaqMan Universal PCR Master Mix II, 2 μl of template cDNA, paired primers and TaqMan MGB probes for targeting gene of interest and exogenously added luciferase RNA, which served as loading control. The PCR amplifications were initiated with an initial denaturing step at 95° C. for 5 min followed by 60 cycles of alternating annealing and elongation at 60° C. for 1 minute each, followed by denaturation at 95° C. for 15 seconds. Each reaction was run in duplicate. To normalize the circulating target gene transcript levels from sample to sample, the exogenously added internal control gene, luciferase was amplified. It should be noted that luciferase gene detection remained proportional to total RNA in each sample. The ratio of the target gene and luciferase was calculated to normalized transcript levels of each target gene. The serum levels of target gene transcripts were determined from standard curves. The results were expressed as copy numbers per 250 μl serum.

Standard Curves. To generate the standard curves, a quantitation protocol for real-time PCR provided by ABI was followed (See “Absolute Quantification Getting Started Guide”, Applied Biosystems, Foster City, Calif.). In brief, plasmid vectors harboring RT-PCR product from total RNA isolated 1205Lu melanoma cells, were prepared by cloning into pCR8/GW/TOPO vector. The copy numbers were calculated from the following formula. m [n][1.096e-21 g/bp], where: n=plasmid size (bp), m=mass. The mass of plasmid DNA needed for given copy number was obtained by multiplying the mass of single plasmid with the copy number of interest. The prepared standard solutions made into small aliquots and stored at −20° C. and thawed only once before use. The standard curves for each gene of interest were obtained using the plasmids containing target genes ranging from 30 to 300,000 copies and primers and probes in duplex with luciferase for internal control. Each sample was run in duplicate. The curves generated for each gene of interest showed a linear relationship between copy numbers and the C_{T} values of real-time PCR for both target genes and luciferase gene.

Statistical Analysis. The probability of metastatic disease versus local disease was modeled as functions of log₁₀ transformations of the individual biomarkers (serum circulating gene transcript levels and serum LDH) using logistic regression (based on weighted least squares estimation). Each biomarker was tested at the alpha=0.05 significance level. For each analyte found to be statistically significant, the odds ratio was estimated indicating the increase in the odds of metastatic disease associated with a log₁₀ increase in the biomarker. The ROC curve (and corresponding AUC) was calculated for each analyte. As a supplemental analysis, each analyte was compared between the metastatic and local disease groups using ANOVA techniques (p-values were calculated based on the log₁₀ transformed data). To preserve a family-wise Type I error rate of 0.05 in the logistic regression and ANOVA analyses of the four test genes (HELLS, TYMS, NCAPH, and BIRC5), Bonferroni-adjusted p-values were calculated.

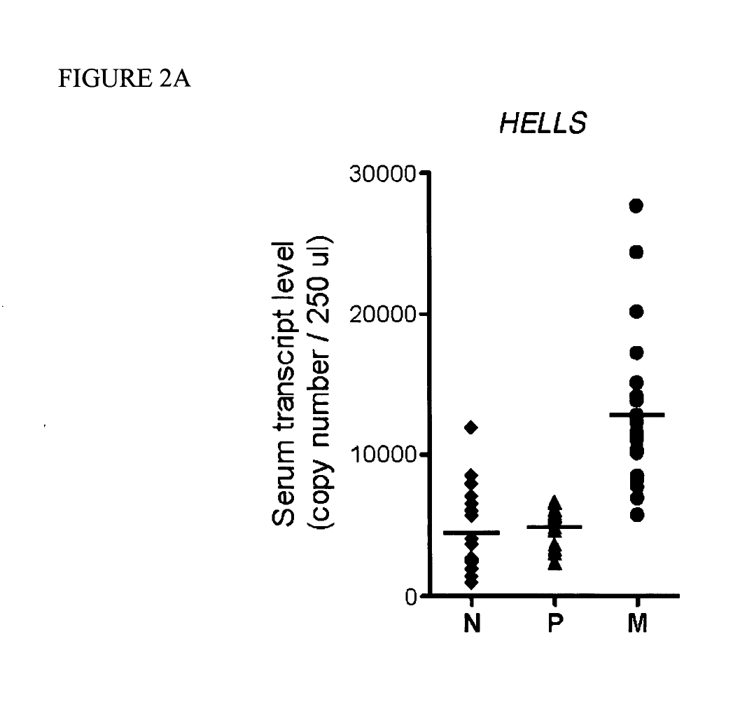

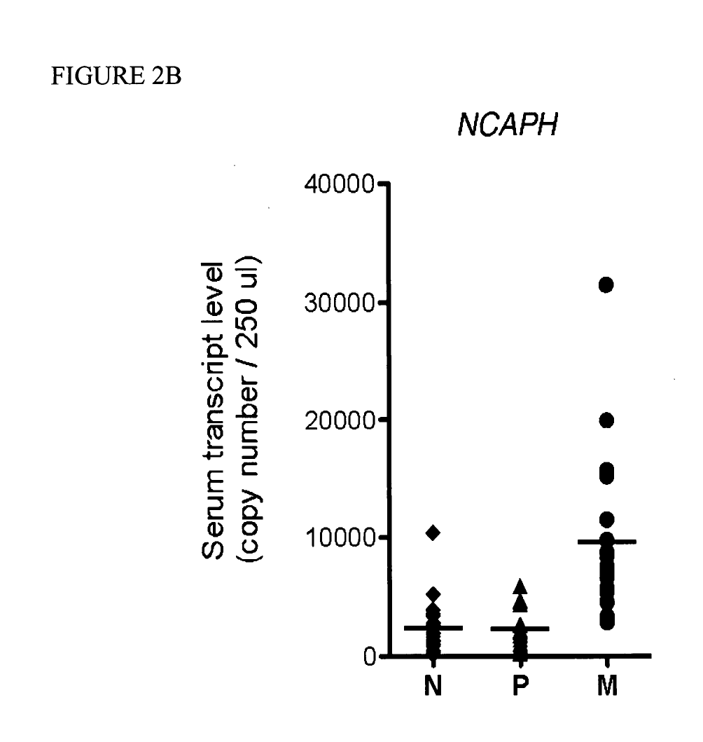

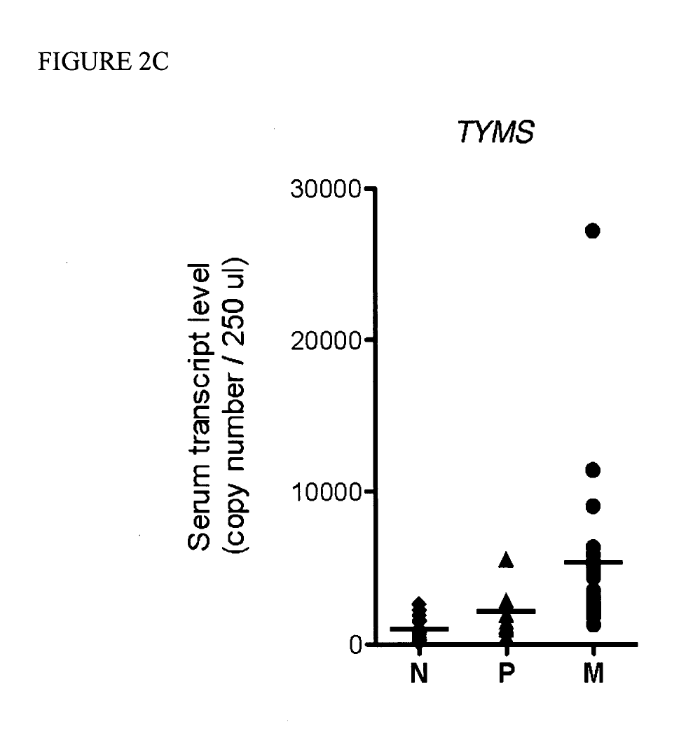

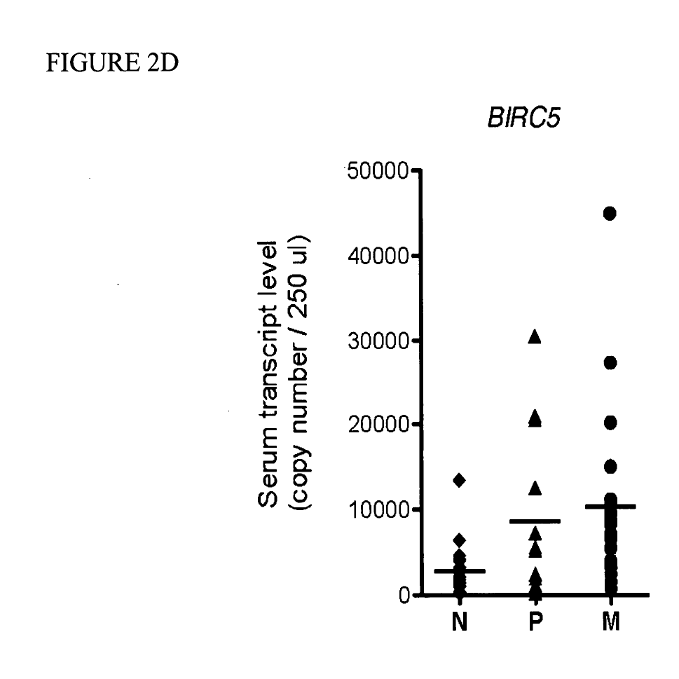

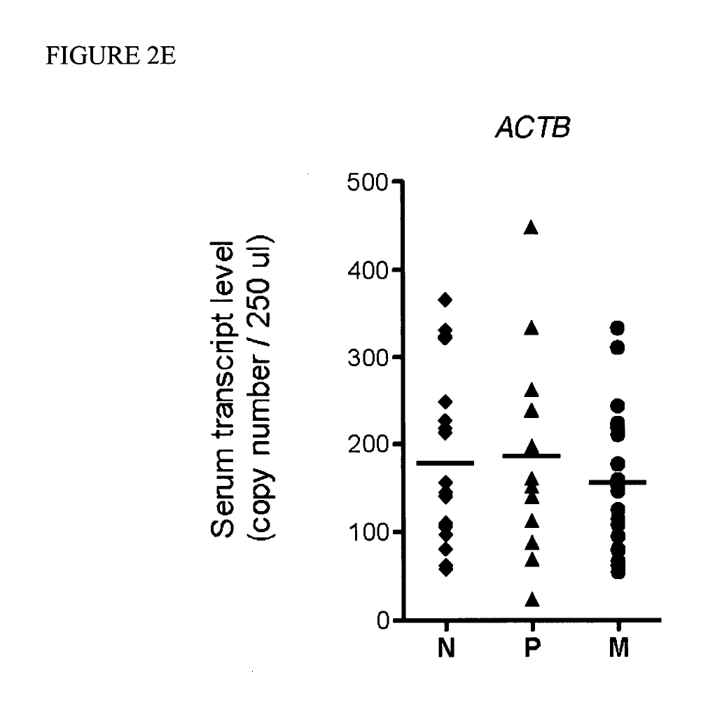

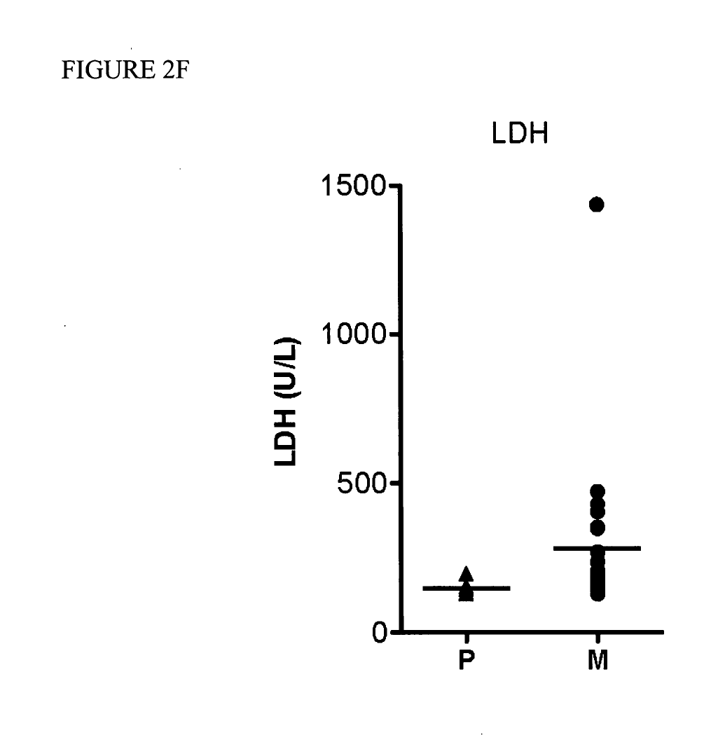

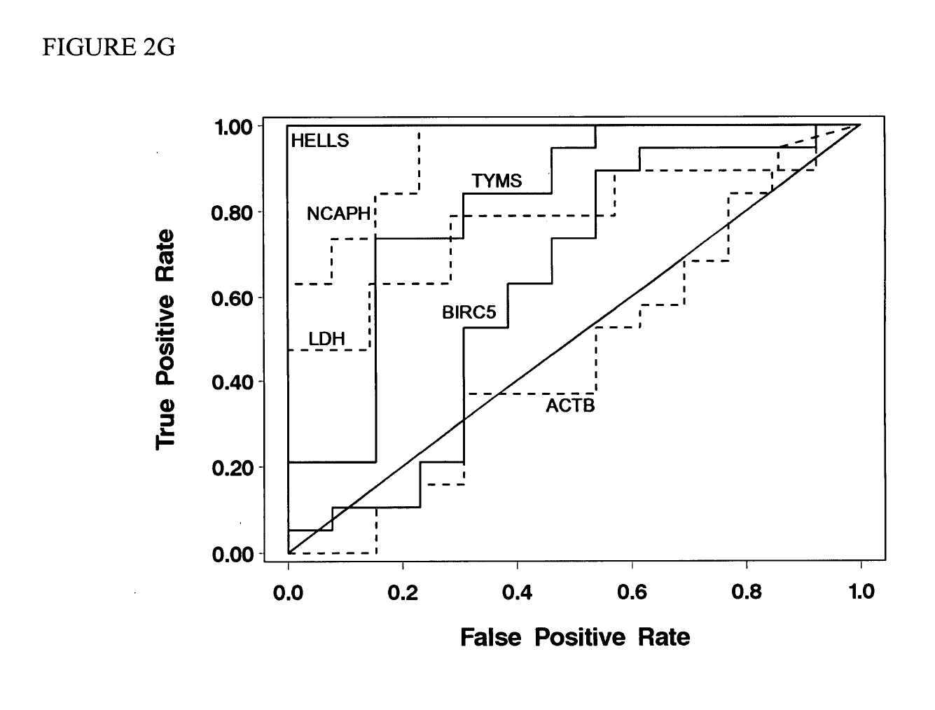

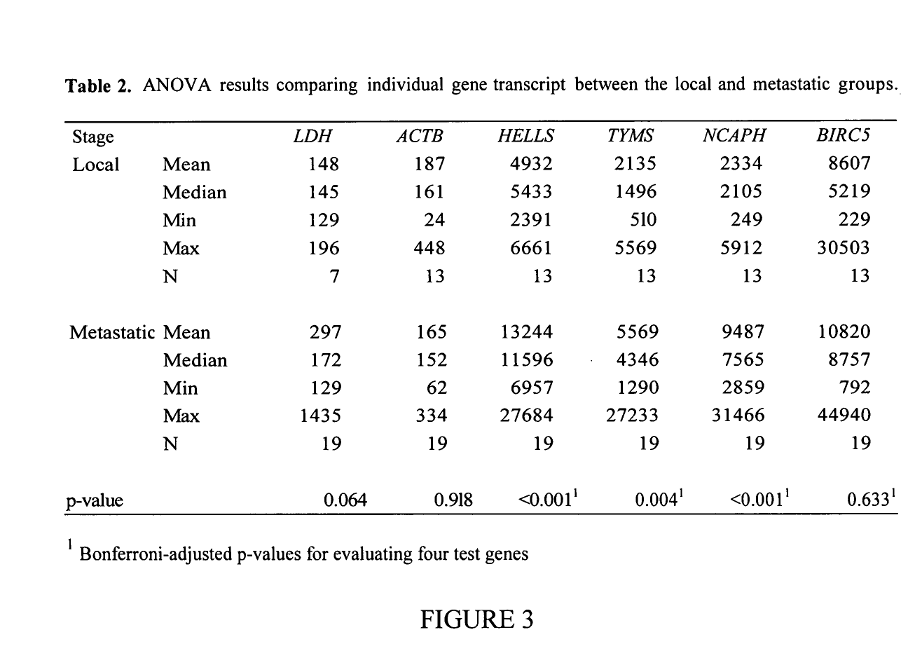

Results. Four genes (HELLS, TYMS, NCAPH, and BIRC5) representing the metastasis-associated gene signature reported by Ryu et al. (Op. cit.) were selected. Serum levels of the gene transcripts circulating in patient blood were quantitatively measured by a duplex real-time q-PCR technique. Serum levels of the selected gene transcripts were compared in two groups of melanoma patients (FIG. 1): group 1 comprised patients with localized melanomas (AJCC stage 0/1) and group 2 comprised patients with distant organ metastasis (AJCC Stage IV). In order to set base levels of the circulating gene transcripts in a normal control population, a group of serum samples from sex- and age-matched healthy controls with no history of illness (average age, 57.8 yr) was included. Key clinical and histopathologic characteristics of the melanoma patients included in this study are summarized in FIG. 1. As shown in FIG. 2A, levels of circulating transcript of the selected genes (HELLS, NCAPH, and TYMS) were clearly different for patients with early stage primary melanomas vs. patients with distant organ metastasis. Serum levels of ACTB (β-actin) gene transcript served as a negative control and showed no differences among the experimental groups (FIG. 2A). Statistical calculations using analysis of variance (ANOVA), as summarized in FIG. 3, demonstrate that the differences were significant for HELLS (p-value<0.001), NCAPH (p-value<0.001), and TYMS (p-value=0.004). However, there were no statistically significant differences between the two groups ACTB (p-value=0.918) (FIG. 3).

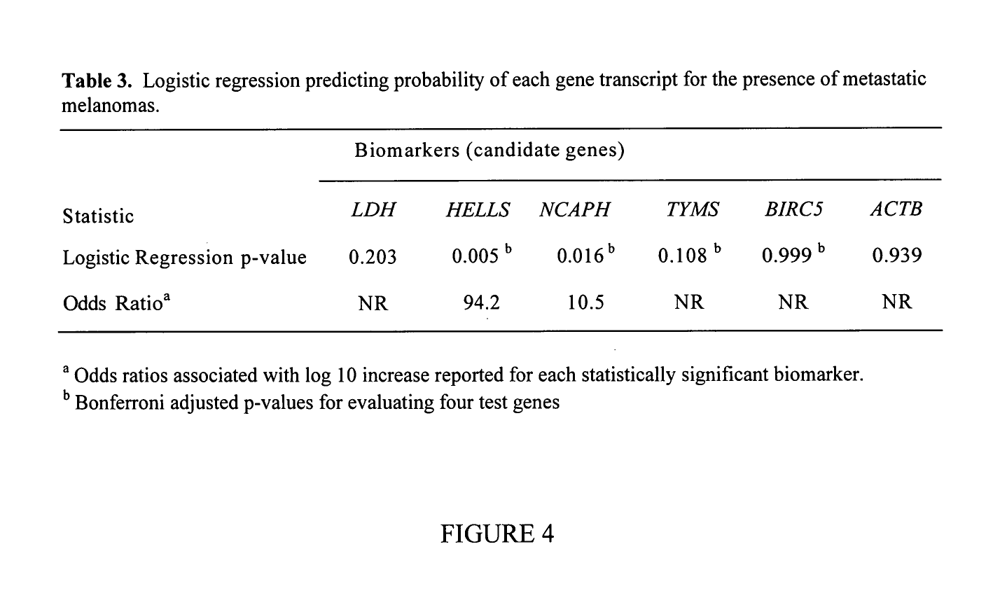

To calculate the predictive value of serum levels of the gene transcripts for presence of metastasis, a logistic regression analysis was performed. The probability of metastasis was increased with increasing serum levels of HELLS and NCAPH but not with TYMS or BIRC5, and the odds ratio of HELLS (94.2) was greater than that of NCAPH (10.5) (FIG. 4). HELLS and NCAPH were included in a multiple logistic regression analysis, and backward elimination was used to determine independent predictors for metastasis. Results showed that only HELLS was independently predictive of metastasis; however, measurement of the combination of HELLS, NCAPH and TYMS were also useful in predicting metastasis.

Serum lactate dehydrogenase (LDH) is a strong prognostic factor in the AJCC staging system for metastatic melanoma, which may influence treatment outcome [Balch et al. Op. cit.; Deichmann, M., Benner, A., Bock, M., Jacket, A., Uhl, K., Waldmann, V., and Naher, H. (1999). S100-Beta, melanoma-inhibiting activity, and lactate dehydrogenase discriminate progressive from nonprogressive American Joint Committee on Cancer stage IV melanoma. J. Clin. Oncol. 17, 1891-6]. Predictivity of the serum levels of the four gene transcripts as metastasis indicators versus that of serum LDH levels was compared by calculating receiver operating characteristic—area under curve (ROC-AUC) values (FIGS. 1B and C). The AUC area was greater for HELLS (1.0) and NCAPH (0.939) than for LDH (0.778). The serum transcript level of HELLS has the highest ROC-AUC value (FIG. 1C). These findings clearly indicated that measurement of the target gene transcript levels in patient serum provided a better discriminating potential than LDH for detection of metastatic melanoma.

The serum biomarkers S100B and melanoma-inhibiting activity (MIA) have been proposed as prognostic biomarkers for melanoma progression, as they have a strong association with overall survival [Bosserhoff, A. K., Kaufmann, M., Kaluza, B., Bartke, 1., Zirngibl, H., Hein, R., Stolz, W., and Buettner, R. (1997). Melanoma-inhibiting activity, a novel serum marker for progression of malignant melanoma. Cancer Res. 57, 3149-53; Von Scoultz, E., Hansson, L. O., Djureen, E., Hansson, J., Karnell, R., Nilsson, B., Stigbrand, T., and Ringborg, U. (1996). Prognostic value of serum analyses of S-100 beta protein in malignant melanoma. Melanoma Res. 6, 133-7]. However, these biomarkers do not have independent prognostic capability when compared to LDH serum concentration [Deichmann et. al. Op. cit.]. Therefore, LDH is currently the only serum marker integrated in the AJCC staging system [Balch et al., Op cit.]. This study clearly demonstrates that measurement of metastasis-associated gene transcript levels in patient serum is superior to serum LDH concentration in discriminating patients with distant organ metastasis (FIG. 2C).

Quantitative detection of cell-free CNAs in patient blood provides a desirable approach to develop diagnostic, prognostic, and therapy response-predicting biomarkers [Swamp and Rajeswari, Op. cit.; Taback and Hoon, Op. cit.]). Since the seminal study demonstrating the feasibility of RT-PCR for detection of melanoma in peripheral blood [Smith, B., Selby, P., Southgate, J., Pittman, K., Bradley, C., and Blair, G. E. (1991). Detection of melanoma cells in peripheral blood by means of reverse transcriptase and polymerase chain reaction. Lancet 338, 1227-9], several reports indicate that the RT-PCR detection of circulating melanoma cells have prognostic significance. The majority of these studies used RT-PCR detection of melanocytic differentiation antigens including tyrosinase, MART-1, and MAGES gene transcripts [Quaglino, P., Savoia, P., Osella-Abate, S., and Bernengo, M. G. (2004). RT-PCR tyrosinase expression in the peripheral blood of melanoma patients. Expert Rev. Molec. Diag. 4, 727-41; Quaglino, P., Osella-Abate, S., Savoia, P., Bernengo, M. G., Cappello, N., and Cavallo, F. (2007). What is the role of sequential reverse-transcriptase polymerase chain reaction analysis of melanoma-specific mRNA in the peripheral blood of melanoma patients? J. Clin. Oncol. 25, 1140-1; author reply, 1141-2]. Notably, Scoggins et al. reported that RT-PCR detection of tyrosinase and other biomarkers in peripheral blood showed significant differences in adverse prognoses [Quaglino et al., Op. cit.; Scoggins, C. R., Ross, M. I., Reintgen, D. S., Noyes, R. D., Goydos, J. S., Beitsch, P. D., Urist, M. M., Ariyan, S., Davidson, B. S., Sussman, J. J., et al. (2006). Prospective multi-institutional study of reverse transcriptase polymerase chain reaction for molecular staging of melanoma. J. Clin. Oncol. 24, 2849-57)]. Here, a class of genes that appear to be functionally associated with melanoma metastasis development was measured quantitatively and have value as a prognostic biomarker for melanoma metastasis. Given this new class of candidate genes' functional association with tumor cell proliferation and metastasis rather than melanin biosynthesis, RT-PCR detection of these novel biomarkers can be used for assessment of prognostic risk in patients with amelanotic melanoma as well.

In summary, circulating mRNA levels of HELLS and NCAPH transcripts in melanoma patient blood were found to be significantly higher in patient with distant organ metastasis compared to those with localized tumors. Area under ROC curves of HELLS and NCAPH transcripts were greater compared to that of serum LDH which is one of the most useful prognostic indicators in metastatic melanoma. By multiple logistic regression analysis, HELLS transcript was identified to be a statistically significant independent biomarker for the presence of distant metastasis. This study shows that elevated serum levels of HELLS gene transcript have use as a biomarker that is more predictive of melanoma metastasis than previously studied markers known in the art, such as LDH.

Although the invention has been described with reference to the above examples, it will be understood that modifications and variations are encompassed within the spirit and scope of the invention. Accordingly, the invention is limited only by the following claims.

We claim:

1. A method of detecting or diagnosing melanoma in a subject comprising:
(a) measuring at least one biomarker in a sample from the subject, wherein the biomarker is selected from the group consisting of: HELLS, NCAPH, TYMS, and BIRC5; and
(b) correlating the measurement of the biomarker with melanoma status, thereby detecting or diagnosing melanoma in the subject.

2. The method of claim 1, wherein the sample is whole blood, serum, plasma, lymphatic fluid, interstitial fluid, cerebrospinal fluid (CSF), seminal fluid, saliva, mucous, sputum, sweat, or urine.

3. The method of claim 1, wherein measuring comprises detecting the presence or absence of the biomarker, quantifying the amount of the biomarker, and qualifying the type of the biomarker.

4. The method of claim 1, wherein the melanoma comprises in situ, radial growth phase, vertical growth phase, recurrent melanoma, or metastatic melanoma.

5. The method of claim 1, wherein the biomarker is HELLS.

6. The method of claim 1, wherein the biomarker is NCAPH.

7. The method of claim 1, wherein at least two biomarkers are measured.

8. The method of claim 1, wherein at least three biomarkers are measured.

9. The method of claim 1, wherein each of HELLS, NCAPH, TYMS and BIRC5 are measured.

10. The method of claim 1, wherein the biomarker is detected by PCR or microarray analysis.

11. The method of claim 1, wherein the biomarker is detected by quantitative real-time RT-PCR.

12. The method of claim 1, wherein the biomarker is detected by immunoassay.

13. The method of claim 1, further comprising: (c) managing subject treatment based on the melanoma status.

14. The method of claim 13, wherein managing subject treatment is selected from ordering further diagnostic tests, administering at least one therapeutic agent, surgery, surgery followed or preceded by administering at least one therapeutic agent, biotherapy, and taking no further action.

15. The method of claim 14, wherein the therapeutic agent is selected from one or more of fotemustine, dacarbazine, interferon, cisplatin, tamoxifen, interleukin-2, interferon alpha, vinblastin, carmubris, avastin, BRAF-kinase inhibitor, CTLA-4 antibody, angiogenesis inhibitors, targeted immunotherapy, or vaccines.

16. The method of claim 13, further comprising: (d) measuring the biomarker after managing subject treatment.

17. The method of claim 1, wherein the melanoma status is selected from one or more of the presence, absence or amount of the biomarker.

18. The method of claim 17, further comprising assessing the status of the melanoma.

19. The method of claim 18, wherein the melanoma status is assessed by one or more of visual examination, tissue sample examination, subject's symptoms, or blood evaluation.

20. The method of claim 1, wherein the subject has previously been diagnosed with melanoma.

21. The method of claim 1, wherein the subject has not been previously diagnosed with melanoma.

22. The method of claim 1, wherein the subject has previously been treated for melanoma.

23. The method of claim 1, wherein the measurement is used to predict the recurrence of melanoma.

24. The method of claim 1, wherein the measurement is used to classify the subject as a low or high risk for melanoma recurrence.

25. The method of claim 1, wherein the correlation is performed by a software classification algorithm.

26. The method of claim 1, further comprising:
correlating the measurement of the biomarker with a melanoma stage.

27. The method of claim 26, wherein the melanoma stage is Stage IV melanoma.

28. A method of determining prognosis of a subject suffering from melanoma comprising: (a) measuring at least one biomarker in a sample from the subject, wherein the biomarker is selected from the group consisting of: HELLS, NCAPH, TYMS, and BIRC5; and (b) correlating the measurement with prognosis of melanoma, thereby determining the prognosis of the subject suffering from melanoma.

29. The method of claim 28, wherein the sample is whole blood, serum, plasma, lymphatic fluid, interstitial fluid, seminal fluid, cerebrospinal fluid (CSF), saliva, mucous, sputum, sweat, or urine.

30. The method of claim 28, wherein measuring comprises detecting the presence or absence of the biomarker, quantifying the amount of biomarker, and qualifying the type of the biomarker.

31. The method of claim 28, wherein the melanoma comprises in situ, radial growth phase, vertical growth phase, or metastatic melanoma.

32. The method of claim 28, wherein the prognosis determines course of treatment.

33. The method of claim 28, wherein the biomarker is HELLS.

34. The method of claim 28, wherein the biomarker is NCAPH.

35. The method of claim 28, wherein at least two biomarkers are measured.

36. The method of claim 28, wherein at least three biomarkers are measured.

37. The method of claim 28, wherein each of HELLS, NCAPH, TYMS and BIRC5 are measured.

38. The method of claim 28, wherein the biomarker is detected by PCR or microarray analysis.

39. The method of claim 28, wherein the biomarker is detected by quantitative real-time RT-PCR.

40. The method of claim 28, further comprising:
(c) managing subject treatment based on the prognosis.

41. The method of claim 40, wherein managing subject treatment is selected from ordering further diagnostic tests, administering at least one therapeutic agent, surgery, surgery followed or preceded by administering at least one therapeutic agent, biotherapy, and taking no further action.

42. The method of claim 41, wherein the therapeutic agent is selected from one or more of fotemustine, dacarbazine, interferon, cisplatin, tamoxifen, interleukin-2, interferon alpha, vinblastin, carmubris, avastin, BRAF-kinase inhibitor, CTLA-4 antibody, angiogenesis inhibitors, targeted immunotherapy, or vaccines.

43. The method of claim 40, further comprising:
(d) measuring the biomarker after subject management.

44. The method of claim 28, wherein the prognosis is determined from one or more of the presence, absence or amount of the biomarker.

45. The method of claim 44, further comprising assessing the prognosis of the melanoma.

46. The method of claim 45, wherein the melanoma prognosis is assessed by one or more of visual examination, tissue sample examination, subject's symptoms, or blood evaluation.

47. The method of claim 28, wherein the subject has previously been diagnosed with melanoma.

48. The method of claim 28, wherein the subject has not been previously diagnosed with melanoma.

49. The method of claim 28, wherein the subject has previously been treated for melanoma.

50. The method of claim 28, wherein the measurement is used to predict the recurrence of melanoma.

51. The method of claim 28, wherein the measurement is used to classify a subject as a low or high risk for melanoma recurrence.

52. The method of claim 28, wherein the correlation is performed by a software classification algorithm.

53. A method of treating melanoma, comprising administering to a subject suffering from melanoma a therapeutically effective amount of a compound capable of modulating the expression or activity of at least one biomarker selected from the group consisting of: HELLS, NCAPH, TYMS, or BIRC5 in the subject.

54. The method of claim 53, wherein the compound is selected from the group consisting of enzyme inhibitors, cytotoxic drugs, cytokines, chemokines, antibodies, a DNA molecule, an RNA molecule, a small molecule, a peptide, and a peptidomimetic.

55. The method of claim 53, wherein the compound modulates the expression or activity of HELLS.

56. The method of claim 53, wherein the compound modulates the expression or activity of NCAPH.

57. The method of claim 53, wherein the compound is an antibody.

58. The method of claim 57, wherein the antibody is selected from the group consisting of: monoclonal, polyclonal, humanized, and chimeric antibodies.

59. The method of claim 58, wherein the antibody is radiolabeled.

60. The method of claim 54, wherein the compound in an inhibitory RNA molecule.

61. The method of claim 60, wherein the inhibitory RNA molecule is one or more siRNAs.

62. A method of determining melanoma status of a subject, comprising:
(a) obtaining a biomarker profile from a sample taken from the subject; and
(b) comparing the subject's biomarker profile to a reference biomarker profile obtained from a reference population, wherein the comparison is capable of classifying the subject as belonging to or not belonging to the reference population; wherein the subject's biomarker profile and the reference biomarker profile comprise at least one biomarker selected from the group consisting of: HELLS, NCAPH, TYMS, and BIRC5.

63. The method of claim 62, wherein the sample is whole blood, serum, plasma, lymphatic fluid, interstitial fluid, seminal fluid, cerebrospinal fluid (CSF), saliva, mucous, sputum, sweat, or urine.

64. The method of claim 62, further comprising repeating the method at least once, wherein the subject's biomarker profile is obtained from a separate sample taken each time the method is repeated.

65. The method of claim 64, wherein samples from the subject are taken about 24 hours apart.

66. The method of claim 62, wherein the comparison of the biomarker profiles determines the melanoma status in the subject with an accuracy of at least about 60% to about 99%.

67. The method of claim 62, wherein the reference biomarker profile is obtained from a population comprising a single subject, at least two subjects, and at least 20 subjects.

68. A method of qualifying cancer status in a subject comprising:
(a) measuring at least one biomarker in a sample from the subject, wherein the biomarker is selected from the group consisting of HELLS, NCAPH, TYMS and BIRC5; and
(b) correlating the measurement with cancer status, thereby qualifying cancer status in the subject.

69. The method of claim 68, wherein the sample is whole blood, serum, plasma, lymphatic fluid, interstitial fluid, seminal fluid, cerebrospinal fluid (CSF), saliva, mucous, sputum, sweat, or urine.

70. The method of claim 68, wherein the biomarker is HELLS.

71. The method of claim 68, wherein the biomarker is NCAPH.

72. The method of claim 68, wherein the cancer is a solid tumor.

73. The method of claim 68, wherein the cancer is a hematological malignancy.

74. The method of claim 68, wherein the correlation is performed by a software classification algorithm.

75. A kit for detecting or diagnosing melanoma, comprising one or more reagents for detecting at least one biomarker selected from the group consisting of HELLS, NCAPH, TYMS and BIRC5, and written instructions for use of the kit for the detection or diagnosis of melanoma.

76. The kit of claim 75, wherein the instructions provide for contacting a sample from a subject with the reagents and detecting the biomarker.

77. The kit of claim 75, wherein the reagents comprise an adsorbent that retains the biomarker.

78. The kit of claim 77, wherein the adsorbent is a single or double stranded oligonucleotide.

79. The kit of claim 75, wherein the biomarker is detected using nucleic acid sequencing or PCR.

80. The kit of claim 75, wherein the biomarker is detected by quantitative real-time RT-PCR.


SEQUENCE LISTING (22 sequences)

SEQ ID NO: 1 - DNA, 3165 bp, Homo Sapiens
ggggatttggctagaaggctgggccggcagcggttgtgaggagttagctcgcggcattgcaggctctgagaggaggggacccggttcccgggtgagtgtccaggcatgccagcggaacggcccgcgggcagcggcggctcggaggctccagcaatggttgaacaactggacactgctgtgattaccccggccatgctagaagaggaagaacagcttgaagctgctggactagagagagagcggaagatgctggaaaaggctcgcatgtcttgggatagagagtcgacagaaattcggtaccgtagacttcaacatttgcttgaaaaaagcaatatatactccaaatttttattgacgaaaatggaacagcaacaattagaggaacagaagaagaaagaaaaattggagagaaaaaaggagtctttaaaagttaaaaagggtaaaaattcaattgatgcaagtgaagagaagccagttatgaggaaaaaaagaggaagagaagatgaatcatacaatatttcagaggtcatgtcaaaagaggaaattttgtctgtggctaaaaaaaataaaaaggagaatgaggatgaaaactcctcctctactaatctctgtgtggaagatcttcagaaaaataaagattcgaatagtataattaaagatagattgtctgaaacggttaggcagaatactaaattcttttttgacccagtccggaagtgtaatggtcagccagtaccttttcaacaaccaaagcacttcactggaggagtgatgcgatggtaccaagtagaaggcatggaatggcttaggatgctttgggaaaatggaattaatggcattttagcagatgaaatgggattgggtaagacagttcagtgcattgctactattgcattgatgattcagagaggagtaccaggaccttttcttgtctgtggccctttgtctacacttcctaactggatggctgaattcaaaagatttacaccagatatccctacaatgttatatcatggaacccaggaggaacgtcaaaaattggtaagaaatatttacaaacggaaagggactttgcagattcatcctgtggtaatcacgtcatttgaaatagccatgagagaccgaaatgcgttacagcattgctattggaaatacttaatagtagatgaaggacacaggattaagaatatgaagtgccgtctaatcagggagttaaaacgattcaatgctgataacaaacttcttttgactggtactcccttgcaaaacaatttatcagaactttggtcattgctaaactttttgttgccagatgtatttgatgacttgaaaagctttgagtcttggtttgacatcactagtctttctgaaactgctgaagatattattgctaaagaaagagaacagaatgtattgcatatgctgcaccagattttaacacctttcttattgagaagactgaagtctgatgttgctcttgaagttcctcctaaacgagaagtagtcgtttatgctccactttcaaagaagcaggagatcttttatacagccattgtgaaccgtacaattgcaaacatgtttggatccagtgagaaagaaacaattgagttaagtcctactggtcgaccaaaacgacgaactagaaaatcaataaattacagcaaaatagatgatttccctaatgaattggaaaaactgatcagtcaaatacagccagaggtggaccgagaaagagctgttgtggaagtgaatatccctgtagaatctgaagttaatctgaagctgcagaatataatgatgctacttcgtaaatgttgtaatcatccatatttgattgaatatcctatagaccctgttacacaagaatttaagatcgatgaagaattggtaacaaattctgggaagttcttgattttggatcgaatgctgccagaactaaaaaaaagaggtcacaaggtgctgcttttttcacaaatgacaagcatgttggacattttgatggattactgccatctcagagatttcaacttcagcaggcttgatgggtccatgtcttactcagagagagaaaaaaacatgcacagcttcaacacggatccagaggtgtttatcttcttagtgagtacacgagctggtggcctgggcattaatctgactgcagcagatacagttatcatttatgatagtgattggaacccccagtcggatcttcaggcccaggatagatgtcatagaattggtcagacaaagccagttgttgtttatcgccttgttacagcaaatactatcgatcagaaaattgtggaaagagcagctgctaaaaggaaactggaaaagttgatcatccataaaaatcatttcaaaggtggtcagtctggattaaatctgtctaagaatttcttagatcctaaggaattaatggaattattaaaatctagagattatgaaagggaaataaaaggatcaagagagaaggtcattagtgataaagatctagagttgttgttagatcgaagtgatcttattgatcaaatgaatgcttcaggaccaattaaagagaagatggggatattcaagatattagaaaattctgaagattccagtcctgaatgtttgttttaaagtggagctcaagaatagcttttaaaagttcttatttacatctagtgatttccctgtattgggtttgaaatactgattgtccacttcaccttttttattatatcagttgacatgtaactagtaccatgcgtacttaaatagatggtaattttctgagccttaccaagaacaaagaagtatccatattaagtttagattttcagttaatttttgagactgagtagtattcttggatacaggctgatgtgtacttaaccacttccagatttatacagtcttcctgtggaagtttagtaaatgtctttttccctcctttcttctagtaatgcagttcatgggctttaggtacttcagttatgaagtaggcttttcatggggagagattgggattatgctctctgttgtttaagaaactgtttgattttagagtctatttctatgagatagtttaccaaataaatgttccttataagatgaaaaaaaaaaaaaaaaaaaaaaaaaaaaaaaaaaaaaaaaaaaaaaaaaaaaaaaaaaaaaaaaaaa

SEQ ID NO: 2 - DNA, 4495 bp, Homo Sapiens
  ref <308>: DATABASE ACCESSION NUMBER: NM_015341
  ref <309>: DATABASE ENTRY DATE: 2011-11-19
  ref <313>: RELEVANT RESIDUES IN SEQ ID NO: (1)..(4495)
gccggcgacgtcacgcggccgttacggcgctcaggcgtctcgacgcgcgcgatttaaaaccagctcaggagacgccaaggaaagatgggacctcccggcccagcactgccagccacaatgaataactcttcttcagagacgcgaggacacccccacagtgcctcctctccttcagagcgtgtgttcccgatgcccctgcccaggaaggcgcctctcaatattcctggcaccccagtcctcgaagactttcctcagaatgacgatgagaaggagcggctgcagcggaggcgctcgagggtctttgatctgcagttcagcactgactcacctcgcttattggcctccccctccagcaggagtattgacatttcagctactatccccaagtttacaaacacgcagattacggaacattactccacctgtatcaaactgtccactgaaaataaaatcactaccaagaatgcttttggtttgcacttgattgattttatgtcagagattcttaaacagaaagacaccgaaccaaccaactttaaagtggctgcgggtactctggatgccagcaccaagatctatgctgtgcgcgtggatgccgtccatgccgatgtatacagagtccttggggggctgggcaaagatgcaccgtctttggaagaagtagaaggccatgttgctgatggaagtgctactgaaatgggaacaaccaaaaaggctgtaaagccaaagaagaagcacttacacagaactattgagcagaacataaacaacctcaatgtctccgaagcagatcggaagtgtgagattgatcccatgtttcagaagacagcagcctcatttgatgagtgcagcacagcaggggtgtttctgtccactctccactgccaggactacagaagtgaactgctgtttccctctgatgtccagactctctccacgggagaacctctcgagttgccagagttaggttgtgtagaaatgacagatttaaaagcgcccttgcagcagtgtgcagaagatcgccagatctgcccttccctggccgggttccagtttacacagtgggacagtgaaacacataatgagtctgtgtcggccctggtagacaagtttaagaagaatgaccaggtatttgacatcaatgctgaagttgacgagagtgactgtggagacttccccgatgggtccctgggggatgactttgatgccaacgatgaacctgaccacaccgcagttggggatcatgaagagttcaggagctggaaggagccctgccaggttcagagctgccaggaagaaatgatttcccttggggatggagacatcaggaccatgtgcccccttctgtctatgaaacctggagaatattcttatttcagtcctcggaccatgtcgatgtgggctggcccggatcactggcgctttaggcctcgacgcaaacaagatgctccttcccaatcagaaaacaaaaagaagagtacaaaaaaagattttgaaattgactttgaagatgatattgactttgatgtatattttagaaaaacaaaggctgctactattctgaccaagtccactttggagaaccagaattggagagctaccacccttcctacagatttcaactacaatgttgacactctggtccagcttcacctcaaaccaggcaccaggttacttaagatggcccagggccatagggtagagactgagcattatgaagaaattgaagactatgattacaacaaccctaacgacacctccaacttttgccctggattacaggctgctgacagtgatgatgaagatttggatgacttatttgtgggacctgttgggaactctgacctctcaccttatccttgccatccacctaagacagcacaacagaatggtgacactccagaagcccaaggattagacatcacaacatatggggagtcaaacttggtagctgagcctcagaaggtaaataaaattgaaattcactatgccaagactgccaaaaagatggacatgaagaaactgaagcagagcatgtggagtctgctgacagcgctctccggaaaggaggcagatgcagaggcaaaccacagggaagctggaaaagaagcggccctggcagaagtggctgacgagaagatgcttagcgggctcacgaaggacctgcagaggagcctgccccctgtcatggctcagaacctctccatacctctggcttttgcctgtctcctacatttagccaatgaaaagaatctaaaactggaaggaacagaggacctctctgatgttcttgtgaggcaaggagattgagttcactatggagaagtcagcagcaggaggcccatcccttactcagttgccgggacatccccagtctcgggggaagaagatgccatgggcttatacccaggctgtagccaactaccaacgtgcctgtttgtttgttgctctttccttctctccatcatagtctgggtgccagcgccctgaagctccgtgctcaactgattaaactttactgccctatggtgaccatctaggagaggggagggcagagggggtgagggtactattctggattgagaaaacctatatccattctttatatcaatgtatagttttagtctcctaaattgatctgttattttccaaactattctcttgtagaaaattttccagtgggcacttaatggtgcccttgaagaacttcctaatccatgtacataaaatacatcatatgtacacttataaatgtatatagaatgctcaaaaataaaattcttaataatagaactggcaaaatatttgagtgtccactagatgagtatcagacctagtccttacccttagggggatgcagtcctggttgttatccaggatacacacctgtcagtataaggcagaagatgcctaagggccaagatggtttgcctcggaggagaatggaagagagagattgctgactggacattcagatgcaagactgggtcctgcttaaatcccaggattctgctggagggagctgatagtgatacttgtcccttctgtacattgcttcatgtagccttctcagcatccctaggagaaacttactattgtgactctcatgttggaggaggaaacggacacccaaggtagaggaacttgcaaaagggcagccggcaaactgtcaggggtggcctgagcctggcaatctgcctccagagtctgctctcggccattgtgctatgtgctacctggataggtcatacaggctcagcagtgggtggagagcagtgctcagatttgtccatctccacagaatgcagcacacacacaaatgtacaagttcttcccctaacctcagaggaataggggaattaactttgcttgcaatttggaacaatattatagatgttgatccaagtagttctgttactggctggtcctggatctctgccagaacacccgtcatcattgactggctaaatagagatcttggatataggccagaagcagtgaagtatataattggaaattgctcctgataataacttccttcttagccaaaaaccacacaaaacaaaaataatcccctccccacaggaatatgctttccaaattgtgtccaaaacattacctgctctgttatattgagaaggttagagacttcagagcatgcttagaaaaagcagtggtgccacaggtgagactccacactctgtcttgctggggctgaagcctccatcactttcccaggccaggttagtgctgggcttcttgctttccttctattcctgagagtagaactggctaagcccattccttccctcagtcagccccacttctctatagtgggttctgggggtggggggctgaattaccagtaaaactagaaagattgggaccaagtgcagtggcccacacctgtaaatcctagcgctttgaaaggaagaggcaggaggattgcttgaagtcaggagttcaagaccagcctgggcaaaatagaaccccatctttaaaaaaaaagtttaaaaattagccaggtacggaggtgtgtgcctgtaatcccagctactcagaaggctgaggtgggataatcacttgagcccaggagtttgaggctgcagtgagctgtgatcacactactgcattccagccaggacaacagagtgagatcctatctcttaaacaaaaaaaaaaactggcgagttcaataccaacttctacaatgaaatccccttccccccacaaccctgcttctcctaagtttccctcattacatggttgctgtgggctatgtgtgctgtggtctgaatgtttgtgtctaaaattcacatgttggtattaagagatagggcctttgggaggtgattaggttatgagggcagatccctcgtgaatgggattagtgctcttataaaagaggcctgaggaagcttgttcgttcctcttgcccttctgccatgtaaggatgcaatgagaaggcaccatctgtgagcaaggagcccctcac

SEQ ID NO: 3 - DNA, 1603 bp, Homo sapiens
  ref <308>: DATABASE ACCESSION NUMBER: NM_001071.2
  ref <309>: DATABASE ENTRY DATE: 2012-02-12
  ref <313>: RELEVANT RESIDUES IN SEQ ID NO: (1)..(1603)
cgggacggccgcgggaaaaggcgcgcggaaggggtcctgccaccgcgccacttggcctgcctccgtcccgccgcgccacttggcctgcctccgtcccgccgcgccacttcgcctgcctccgtcccccgcccgccgcgccatgcctgtggccggctcggagctgccgcgccggcccttgccccccgccgcacaggagcgggacgccgagccgcgtccgccgcacggggagctgcagtacctggggcagatccaacacatcctccgctgcggcgtcaggaaggacgaccgcacgggcaccggcaccctgtcggtattcggcatgcaggcgcgctacagcctgagagatgaattccctctgctgacaaccaaacgtgtgttctggaagggtgttttggaggagttgctgtggtttatcaagggatccacaaatgctaaagagctgtcttccaagggagtgaaaatctgggatgccaatggatcccgagactttttggacagcctgggattctccaccagagaagaaggggacttgggcccagtttatggcttccagtggaggcattttggggcagaatacagagatatggaatcagattattcaggacagggagttgaccaactgcaaagagtgattgacaccatcaaaaccaaccctgacgacagaagaatcatcatgtgcgcttggaatccaagagatcttcctctgatggcgctgcctccatgccatgccctctgccagttctatgtggtgaacagtgagctgtcctgccagctgtaccagagatcgggagacatgggcctcggtgtgcctttcaacatcgccagctacgccctgctcacgtacatgattgcgcacatcacgggcctgaagccaggtgactttatacacactttgggagatgcacatatttacctgaatcacatcgagccactgaaaattcagcttcagcgagaacccagacctttcccaaagctcaggattcttcgaaaagttgagaaaattgatgacttcaaagctgaagactttcagattgaagggtacaatccgcatccaactattaaaatggaaatggctgtttagggtgctttcaaaggagctcgaaggatattgtcagtctttaggggttgggctggatgccgaggtaaaagttctttttgctctaaaagaaaaaggaactaggtcaaaaatctgtccgtgacctatcagttattaatttttaaggatgttgccactggcaaatgtaactgtgccagttctttccataataaaaggctttgagttaactcactgagggtatctgacaatgctgaggttatgaacaaagtgaggagaatgaaatgtatgtgctcttagcaaaaacatgtatgtgcatttcaatcccacgtacttataaagaaggttggtgaatttcacaagctatttttggaatatttttagaatattttaagaatttcacaagctattccctcaaatctgagggagctgagtaacaccatcgatcatgatgtagagtgtggttatgaactttaaagttatagttgttttatatgttgctataataaagaagtgttctgcattcgtcaaaaaaaaaaaaaaaaaaaa

SEQ ID NO: 4 - DNA, 2537 bp, Homo sapiens
  ref <308>: DATABASE ACCESSION NUMBER: NM_001012270.1
  ref <309>: DATABASE ENTRY DATE: 2012-02-26
  ref <313>: RELEVANT RESIDUES IN SEQ ID NO: (1)..(2537)
cccagaaggccgcggggggtggaccgcctaagagggcgtgcgctcccgacatgccccgcggcgcgccattaaccgccagatttgaatcgcgggacccgttggcagaggtggcggcggcggcatgggtgccccgacgttgccccctgcctggcagccctttctcaaggaccaccgcatctctacattcaagaactggcccttcttggagggctgcgcctgcaccccggagcggatggccgaggctggcttcatccactgccccactgagaacgagccagacttggcccagtgtttcttctgcttcaaggagctggaaggctgggagccagatgacgaccccatgcaaaggaaaccaacaataagaagaaagaatttgaggaaactgcggagaaagtgcgccgtgccatcgagcagctggctgccatggattgaggcctctggccggagctgcctggtcccagagtggctgcaccacttccagggtttattccctggtgccaccagccttcctgtgggccccttagcaatgtcttaggaaaggagatcaacattttcaaattagatgtttcaactgtgctcttgttttgtcttgaaagtggcaccagaggtgcttctgcctgtgcagcgggtgctgctggtaacagtggctgcttctctctctctctctcttttttgggggctcatttttgctgttttgattcccgggcttaccaggtgagaagtgagggaggaagaaggcagtgtcccttttgctagagctgacagctttgttcgcgtgggcagagccttccacagtgaatgtgtctggacctcatgttgttgaggctgtcacagtcctgagtgtggacttggcaggtgcctgttgaatctgagctgcaggttccttatctgtcacacctgtgcctcctcagaggacagtttttttgttgttgtgtttttttgtttttttttttttggtagatgcatgacttgtgtgtgatgagagaatggagacagagtccctggctcctctactgtttaacaacatggctttcttattttgtttgaattgttaattcacagaatagcacaaactacaattaaaactaagcacaaagccattctaagtcattggggaaacggggtgaacttcaggtggatgaggagacagaatagagtgataggaagcgtctggcagatactccttttgccactgctgtgtgattagacaggcccagtgagccgcggggcacatgctggccgctcctccctcagaaaaaggcagtggcctaaatcctttttaaatgacttggctcgatgctgtgggggactggctgggctgctgcaggccgtgtgtctgtcagcccaaccttcacatctgtcacgttctccacacgggggagagacgcagtccgcccaggtccccgctttctttggaggcagcagctcccgcagggctgaagtctggcgtaagatgatggatttgattcgccctcctccctgtcatagagctgcagggtggattgttacagcttcgctggaaacctctggaggtcatctcggctgttcctgagaaataaaaagcctgtcatttcaaacactgctgtggaccctactgggtttttaaaatattgtcagtttttcatcgtcgtccctagcctgccaacagccatctgcccagacagccgcagtgaggatgagcgtcctggcagagacgcagttgtctctgggcgcttgccagagccacgaaccccagacctgtttgtatcatccgggctccttccgggcagaaacaactgaaaatgcacttcagacccacttatttctgccacatctgagtcggcctgagatagacttttccctctaaactgggagaatatcacagtggtttttgttagcagaaaatgcactccagcctctgtactcatctaagctgcttatttttgatatttgtgtcagtctgtaaatggatacttcactttaataactgttgcttagtaattggctttgtagagaagctggaaaaaaatggttttgtcttcaactcctttgcatgccaggcggtgatgtggatctcggcttctgtgagcctgtgctgtgggcagggctgagctggagccgcccctctcagcccgcctgccacggcctttccttaaaggccatccttaaaaccagaccctcatggctaccagcacctgaaagcttcctcgacatctgttaataaagccgtaggcccttgtctaagtgcaaccgcctagactttctttcagatacatgtccacatgtccatttttcaggttctctaagttggagtggagtctgggaagggttgtgaatgaggcttctgggctatgggtgaggttccaatggcaggttagagcccctcgggccaactgccatcctggaaagtagagacagcagtgcccgctgcccagaagagaccagcaagccaaactggagcccccattgcaggctgtcgccatgtggaaagagtaactcacaattgccaataaagtctcatgtggttttatctaaaaaaaaaaaaaaaaaaaaaaaaa

SEQ ID NO: 5 - DNA, 26 bp, Artificial Sequence
  Artificial sequence primer for Homo sapiens HELLS gene
ccctcctttcttctagtaatgcagtt

SEQ ID NO: 6 - DNA, 21 bp, Artificial Sequence
  Artificial sequence primer for Homo sapiens HELLS gene
cccaatctctccccatgaaaa

SEQ ID NO: 7 - DNA, 18 bp, Artificial Sequence
  Artificial sequence probe for Homo sapiens HELLS gene
atgggctttaggtacttc

SEQ ID NO: 8 - DNA, 20 bp, Artificial Sequence
  Artificial sequence primer for Homo sapiens TYMS gene
ttttggacagcctgggattc

SEQ ID NO: 9 - DNA, 20 bp, Artificial Sequence
  Artificial sequence primer for Homo sapiens TYMS gene
gccataaactgggcccaagt

SEQ ID NO: 10 - DNA, 15 bp, Artificial Sequence
  Artificial sequence probe for Homo sapiens TYMS gene
ccaccagagaagaag

SEQ ID NO: 11 - DNA, 20 bp, Artificial Sequence
  Artificial sequence primer for Homo sapiens NCAPH gene
ctggattacaggctgctgac

SEQ ID NO: 12 - DNA, 20 bp, Artificial Sequence
  Artificial sequence primer for Homo sapiens NCAPH gene
ggtcagagttcccaacaggt

SEQ ID NO: 13 - DNA, 20 bp, Artificial Sequence
  Artificial sequence probe for Homo sapiens NCAPH gene
cacaaataagtcatccaaat

SEQ ID NO: 14 - DNA, 20 bp, Artificial Sequence
  Artificial sequence primer for Homo sapiens BIRC5 gene
cgctttcctttctgtcaaga

SEQ ID NO: 15 - DNA, 20 bp, Artificial Sequence
  Artificial sequence primer for Homo sapiens BIRC5 gene
cttggctctttctctgtcca

SEQ ID NO: 16 - DNA, 20 bp, Artificial Sequence
  Artificial sequence probe for Homo sapiens BIRC5 gene
agtttgaagaattaaccctt

SEQ ID NO: 17 - DNA, 20 bp, Artificial Sequence
  Artificial sequence primer for Homo sapiens ACTB gene
ctggaacggtgaaggtgaca

SEQ ID NO: 18 - DNA, 20 bp, Artificial Sequence
  Artificial sequence primer for Homo sapiens ACTB gene
cggccacattgtgaactttg

SEQ ID NO: 19 - DNA, 15 bp, Artificial Sequence
  Artificial sequence probe for Homo sapiens ACTB gene
tcggttggagcgagc

SEQ ID NO: 20 - DNA, 22 bp, Artificial Sequence
  Artificial Primer for luciferase gene from species Photinus pyralis
cgtacgtgatgttcacctcgat

SEQ ID NO: 21 - DNA, 17 bp, Artificial Sequence
  Artificial Primer for luciferase gene from species Photinus pyralis
cgccctggttcctggaa

SEQ ID NO: 22 - DNA, 16 bp, Artificial Sequence
  Artificial probefor luciferase gene from species Photinus pyralis
tgcatctgtaaaagca